SEQUENCE LISTING (10 sequences)

SEQ ID NO: 1 - DNA, 29 bp, Artificial Sequence
  Synthetic sequence
gactgtacagggctgctcacatattctgg

SEQ ID NO: 2 - DNA, 30 bp, Artificial Sequence
  Synthetic sequence
ctgtgtacagagtgtatgtggctgtggccc

SEQ ID NO: 3 - DNA, 20 bp, Artificial Sequence
  Synthetic sequence
tccacacctgcctagtctcg

SEQ ID NO: 4 - DNA, 20 bp, Artificial Sequence
  Synthetic sequence
gtgccagaggcagttgagtt

SEQ ID NO: 5 - DNA, 55 bp, Artificial Sequence
  Synthetic sequence
atatcatcgatcgcgatgcattaattaagcggccgaagactgtaggtcctgaccc

SEQ ID NO: 6 - DNA, 33 bp, Artificial Sequence
  Synthetic sequence
ggtggcgccgcttccttgattctcctgggccac

SEQ ID NO: 7 - DNA, 33 bp, Artificial Sequence
  Synthetic sequence
gcccaggagaatcaaggaagcggcgccaccaat

SEQ ID NO: 8 - DNA, 41 bp, Artificial Sequence
  Synthetic sequence
ggagaaggatactcatgtcagcttggtcttttctttgatcc

SEQ ID NO: 9 - DNA, 38 bp, Artificial Sequence
  Synthetic sequence
aagaccaagctgacatgagtatccttctcctgtcctgc

SEQ ID NO: 10 - DNA, 54 bp, Artificial Sequence
  Synthetic sequence
acgtaacagatctgatatcacgcgtgtacactagtgccctgctgagtccctgag

(19) United States
(12) Patent Application Publication
(10) Pub. No.: US 2022/0275403 A1
(43) Pub. Date: Sep. 1, 2022

(54) Methods And Compositions For Enhancing AAV-Mediated Homologous Recombination Using Ribonucleotide Reductase Inhibitors

(71) Applicant: The Board of Trustees of the Leland Stanford Junior University, Stanford, CA (US)

(72) Inventors: Mark A. Kay, Los Altos, CA (US); Shinnosuke Tsuji, Redwood City, CA (US)

(21) Appl. No.: 17/636,010

(22) PCT Filed: Aug. 17, 2020
(86) PCT No.: PCT/US2020/046700

§ 371 Date: Feb. 16, 2022

Related U.S. Application Data
(60) Provisional application No. 63/029,248, filed on May 22, 2020.
(60) Provisional application No. 62/888,934, filed on Aug. 19, 2019.

(51) Int. Cl. C12N 15/90; C12N 15/86; A61K 48/00; C12N 15/113; A61K 31/7076; A61K 31/17; A61K 31/7068

(52) U.S. Cl. CPC C12N 15/907; C12N 15/86; A61K 48/0008; C12N 15/1137; A61K 31/7076; A61K 31/17; A61K 31/7068; C12N 2750/14143; C12N 2310/14

(57) ABSTRACT

The present disclosure provides methods and compositions for facilitating efficient adeno-associated virus (AAV)-based homologous recombination (HR). Subject methods include a step of contacting a cell (e.g., a population of cells) with a ribonucleotide reductase inhibitor, which provides for increased HR efficiency compared to performing HR in the absence of the inhibitor. The cell is also contacted with a recombinant adeno-associated vims (rAAV) that includes a donor DNA having a sequence cassette (i.e., a nucleotide sequence of interest) flanked by homology arms that facilitate integration of the sequence cassette into a target genomic locus via HR.

### CROSS REFERENCE

This application claims the benefit of U.S. Provisional Patent Application Nos. 62/888/934 filed Aug. 19, 2019 and 63/029,248 filed May 22, 2020, each of which applications is incorporated herein by reference in its entirety

### STATEMENT REGARDING FEDERALLY SPONSORED RESEARCH

This invention was made with Government support under contract HL064274 awarded by the National Institutes of Health. The Government has certain rights in the invention.

### INTRODUCTION

Site-specific manipulation of the genome is a desirable goal for many applications in medicine, biotechnology, and biological research. In recent years much effort has been made to develop new technologies for gene targeting in mitotic and post mitotic cells. Adeno-associated virus (AAV)-based genome editing (e.g., non-nuclease mediated AAV homologous recombination) is a promising technology in many venues of gene-based therapeutics. Genome editing technologies that take advantage of homologous recombination (HR) have been limited in application because of the relatively low efficiency of HR both in vitro and in vivo. Provided herein are compositions and methods that address these limitations

### SUMMARY

The present disclosure provides methods and compositions for facilitating efficient adeno-associated virus (AAV)-based homologous recombination (HR). Subject methods include a step of contacting a cell (e.g., a population of cells) with a ribonucleotide reductase inhibitor, which provides for increased HR efficiency compared to performing HR in the absence of the inhibitor. The cell is also contacted with a recombinant adeno-associated virus (rAAV) that includes a donor DNA having a sequence cassette (i.e., a nucleotide sequence of interest) flanked by homology arms that facilitate integration of the sequence cassette into a target genomic locus (via HR)—for example the homology arms are homologous to sequences flanking an integration site in the targeted genomic locus. The sequence cassette includes a transgene sequence (e.g., a sequence that encodes a protein of interest such as a therapeutic protein, a non-coding RNA such as an siRNA, and the like). Thus, the subject methods provide for efficient integration of the sequence cassette, and therefore the transgene sequence, into a genomic locus via AAV delivery and HR.

In some cases, the donor DNA is configured such that the transgene sequence of interest will be operably linked to the promoter at the target locus upon insertion into the target locus. In some embodiments, the sequence cassette of the donor DNA includes a promoter operably linked to the transgene sequence of interest such that upon integration into the genome, expression of the transgene sequence will remain under the control of the promoter from the sequence cassette of the donor DNA. In some embodiments, the sequence cassette comprises two or more (e.g., 3 or more, 4 or more, or 5 or more) transgene sequences.

In some cases, the sequence cassette integrates into the genomic locus such that after integration, the transgene sequence and the endogenous gene are both expressed under control of the endogenous gene's promoter without significantly disrupting expression of the endogenous gene. In some cases, the sequence cassette includes a sequence, positioned 5′ or 3′ to the transgene sequence, that promotes production of two independent gene products upon integration of the sequence cassette into the genomic locus. Examples of such a sequence include but are not limited a sequence that encodes a 2A peptide, an IRES, an intein, a recognition sequence for a site specific protease, a cleavable linker that is cleaved as part of the coagulation cascade, a factor XI cleavage site, or an intronic splice donor/splice acceptor sequence.

In some cases, a subject method does not include delivering a nuclease or nucleic acid encoding a nuclease to the population of cells (e.g., in some cases the HR proceeds without a prior cleavage step performed by a site-specific nuclease). In some cases, a subject method does include a step of delivering a site-specific nuclease (e.g., a ZFN, a TALEN, a CRISPR/Cas effector protein) or a nucleic acid encoding the site-specific nuclease to the population of cells.

In some cases the contacted population of cells is in vitro. In some cases the contacted population of cells is in vivo. In some cases, the RNR inhibitor includes one or more compounds selected from the group consisting of: hydroxyurea (HU), motexafin gadolinium, fludarabine (Flu), cladribine, gemcitabine, tezacitabine, triapine, and gallium maltolate. In some cases, the RNR inhibitor includes fludarabine, HU, and/or gemcitabine. In some cases, the RNR inhibitor includes fludarabine. In some cases, the RNR inhibitor includes HU. In some cases, the RNR inhibitor includes gemcitabine. In some cases, the sequence cassette integrates into two chromosomes such that the integration is homozygotic.

Reagents, compositions, and kits/systems that find use in practicing the subject methods are also provided.

### BRIEF DESCRIPTION OF THE DRAWINGS

FIG. 1 Contact with Hydroxyurea (HU) (a ribonucleotide reductase (RNR) inhibitor) significantly enhanced AAV-HR efficiency in HeLa cells infected with rAAV-GAPDH/GFP at 50,000 MOI: HeLa cells were pretreated with HU for 12 hours and then infected with rAAV-GAPDH/GFP for 48 hours. The number of GFP positive cells were measured by FACS analysis.

FIG. 2 HU treatment significantly enhanced AAV-HR efficiency in HeLa cells infected with rAAV-GAPDH/GFP at various multiplicity of infection (MOI). HeLa cells were pretreated with HU for 12 hours and then infected with rAAV-GAPDH/GFP at each MOT indicated in the figure for 48 hours. The number of GFP positive cells were measured by FACS analysis.

FIG. 3 Persistence of enhanced AAV-HR efficiency by HU treatment. HeLa cells were pretreated with HU for 12 hours and then infected with rAAV-GAPDH/GFP. The number of GFP positive cells were measured by FACS analysis at indicated time points.

FIG. 4 Like HU, fludarabine (another RNR inhibitor) significantly enhanced AAV-HR efficiency cultured cells (Hun7 cells).

FIG. 5 Depicts results from in vivo delivery of AAV-based HR in mice. A schematic depicts the injection schedule. rAAV was used to deliver a donor sequence encoding human coagulation factor IX (hF9) into the genomic Albumin (Alb) locus. Plasma hF9 expression in the mouse liver was measured as an assay to detect integration and expression of the integrated donor sequence. Fludarabine (Flu) treatment increased hF9 expression in mice injected with a rAAV-A1bF9 targeting vector. Fludarabine treatment continuously enhanced hF9 expression in mice following rAAV8 with Alb/F9 targeting genome. Experimental plan and time schedule: each drug was given three sequential days by ip injection and AAV was injected at first day with 1e11 vector genome (vg)/g body weight. Serum samples were collected at each time point and hF9 levels were measured by ELISA. Body weight of each mouse were measured to monitor the toxic effect of each drug. These data showed that RNR inhibition (e.g., Fludarabine treatment) can increase hF9 expression (relative to controls) without any body weight change (relative to controls)—over an extended period of time.

FIG. 6 Depicts a table from Anu Marahatta et al., (2015), which demonstrated that the concentration of HU—after in vivo HU delivery—is low in the liver of mice relative to its concentration in other organs/tissues.

FIG. 7 Gemcitabine (another RNR inhibitor) treatment also enhanced AAV-HR efficiency, but teniposide (a topoisomerase inhibitor) showed no effect. Huh7 cells were pretreated with indicated drugs for 16 hours and drugs were washed out followed by rAAV infection (GAPDH/GFP targeting vector). Cells were passaged every 2-3 days and FACS analysis were performed 14 days after infection. GFP positive cells were detected by FACS analysis. The data showed that—like other RNR inhibitors—gemcitabine increased the amount of the GFP positive fraction. On the other hand, a topoisomerase inhibitor, teniposide, did not exhibit an effect. These data suggest that inhibition of RNR in general (e.g., via any convenient method) enhances AAV-HR efficiency.

FIG. 8A-8E Fludarabine administration increased the efficiency of gene targeting in hepatocytes of mice Mice from FIG. 5 were sacrificed on the last day of blood collection for collection of liver tissues. FIG. 8A, Genomic DNA was extracted from liver tissues 65 days after Alb-P2A-hF9 vector injection and qPCR was performed to quantify the amount of total AAV genomes. Actb primers were used for quantification of the number of diploid genomes. Error bars represent s.d.; n=5. FIG. 8B, A schematic representing the gene targeting Alb-P2A-hF9 vector. Exon-intron structure and the positions of qPCR primer pairs used for C-E are indicated. FIG. 8C-8E, Total RNA was also extracted from these liver tissues and qPCRs were performed to quantify the expression levels of on-target integrated Alb-P2A-hF9 fusion mRNA (primers Fw1 and Rv2) (C), total hF9 mRNA (Fw2 and Rv3) (D) and endogenous Albumin mRNA (Fw1 and Rv1) (E). Actb mRNA was used for normalization and data is shown as relative expression to the PBS-treated group. Error bars represent s.d.; n=5.

FIG. 9 Fludarabine administration increased the number of hF9 positive hepatocytes of mice Detection of hF9 mRNA (red) in liver sections using RNAscope in situ hybridization. Liver sections of mice from non-injected group, PBS-treated group and Flu-treated group were used for hybridization and counterstained with hematoxylin. Representative images from each injected group are shown and images were taken with 5× or 20× objective with identical exposure and settings.

FIG. 10 The effect of different Flu dosing regimens on gene targeting efficiency. Flu (125 mg/kg) was administered i.p. 3 times per day for 1, 3, or 5 sequential days. Mice were i.v. injected at Day1, immediately after the 2nd administration of Flu, with the Alb-P2A-hF9 targeting vector (1.0×10″ vg/mouse). Blood was collected 2 months after AAV injection and hF9 protein levels were determined via ELISA. Error bars represent s.d.; n=4

FIG. 11A-11B. Delayed dosing of fludarabine failed to increase the efficiency of gene targeting in vivo Mice were i.v. injected with rAAV8 Alb-P2A-hF9 targeting vector (1.0×10″ vg/mouse). Four weeks later, PBS (control) or fludarabine (125 mg/kg) was administered i.p. three times per day for three days. n=2 per group. Serum samples were collected before (Day 22, A) and after (Day 54, B) fludarabine administration and hF9 protein levels were determined by ELISA assay.

FIG. 12 Fludarabine administration increased the efficiency of gene targeting at the ApoE locus in vivo Mice were treated with Flu or PBS, as described before, and injected with 1.0×10″ vg of a gene targeting vector, rAAV8-ApoE-P2A-hF9, targeting the murine ApoE locus. Serum was collected at various times across a nearly 60-day time course and hF9 protein levels were determined. Error bars represent s.e.; n=4

FIG. 13A-13G Fludarabine administration transiently inhibits S-phase cell cycle progression and incurs a DNA damage response in mice FIG. 13A, A schematic displaying injection schedules for assessing cell proliferation and DNA damage response in mouse livers. Mice were i.p. injected with BrdU (200 mg/kg) in PBS once per day for three days to label proliferating hepatocytes. Some mice were simultaneously injected with Flu (125 mg/kg, three times per day for three days) (group 2), while the final group were treated with the same Flu injection schedule prior to the three days of BrdU injection (group 3). n=3. FIG. 13B, Six hours after the last injection, mice were sacrificed and liver tissue sections were used for immune-staining using an anti-BrdU antibody. Representative images from each injected group are shown with BrdU labeled nuclei (red) and a DAPI counterstain (blue). All images were taken with 20× objective with identical exposure and settings. FIG. 13C, Images of BrdU labeled nuclei were quantified from each group and displayed as number of BrdU+ nuclei per field of view. Images used for quantification are from two or more slides per animal, three animals per group, and two or more independent stains. FIG. 13D, Liver tissue sections from the same animals were also stained for the DNA damage response marker P Ser139 γH2AX. Representative images are shown with P Ser139 γH2AX (red) and DAPI (blue). FIG. 13E, Images of P Ser139 γH2AX nuclei were quantified from each group and displayed as the percentage of P Ser139 γH2AX+ nuclei out of all nuclei. Images used for quantification are from two or more slides per animal, three animals per group, and two or more independent stains. FIGS. 13F and 13G, Liver tissue lysates from the same mice were used for Western blotting of P Ser139 γH2AX, and α-tubulin as a loading control. Each lane is data from one animal except for DEN-treatment, which is data from a single mouse sample. The graph shows image analysis quantification of the P Ser139 γH2AX band intensity in the Western normalized to α-tubulin

FIG. 14A-14E The effect of DEN administration on the efficiency of gene targeting in mice liver FIG. 14A, DEN (10 or 30 mg/kg) was administered through a single i.p. injection per day for three days. Mice were also injected i.v. with rAAV8 packaged Alb-P2A-hF9 gene targeting vector (1.0×10″ vg/mouse) on Day 1. Body weight was measured at the indicated time points. Error bars represent s.e.; n=4. FIG. 14B, Serum hF9 protein levels in each treatment group was determined by ELISA. Error bars represent s.e.; n=4. FIG. 14C-14E, Total RNA was extracted from liver tissues and qPCR was performed to quantify the expression levels of (C) endogenous Albumin mRNA, (D) total hF9 mRNA and (E) on-target integration derivedAlb-P2A-hF9 fusion mRNA. Actb mRNA was used for normalization and each data is shown as relative expression to the vehicle (saline)-injected control group. Error bars represent s.e.; n=4.

FIG. 15A-15B Fludarabine administration increased the efficiency of CRISPR/Cas9-mediated gene editing in vivo Mice were treated with Flu or PBS, as described before, and injected with 6.0×10¹² vg/kg of SaCas9-sgRNA8 vectors together with 6.0×10¹² vg/kg or 3.0×10¹³ vg/kg of rAAV8-Alb-P2A-GFP vectors. 2 weeks after the injection, mice were sacrificed, and liver tissue sections were used for immuno-staining using an anti-GFP antibody. FIG. 15A, Representative images are shown with GFP labeled cells (green) and a DAPI counterstain (blue). All images were taken with identical exposure and settings. FIG. 15B, Images of GFP staining were quantified from each group and displayed as the percentage of GFP+ cells out of all nuclei. Error bars represent s.e.; n=4-5.

### DEFINITIONS

A DNA sequence that “encodes” a particular RNA is a DNA nucleic acid sequence that is transcribed into RNA. A DNA polynucleotide may encode an RNA (mRNA) that is translated into protein, or a DNA polynucleotide may encode an RNA that is not translated into protein (e.g. tRNA, rRNA, or a guide RNA; also called “non-coding” RNA or “ncRNA”).

A “protein coding sequence” or a sequence that encodes a particular protein, is a nucleic acid sequence that is transcribed into mRNA (in the case of DNA) and is translated (in the case of mRNA) into a polypeptide in vitro or in vivo when placed under the control of appropriate regulatory sequences. The boundaries of the coding sequence are determined by a start codon at the 5′ terminus (N-terminus) and a translation stop nonsense codon at the 3′ terminus (C-terminus). A coding sequence can include, but is not limited to, cDNA from prokaryotic or eukaryotic mRNA, genomic DNA sequences from prokaryotic or eukaryotic DNA, and synthetic nucleic acids. A transcription termination sequence will usually be located 3′ to the coding sequence.

As used herein, a “promoter sequence” is a DNA regulatory region capable of binding RNA polymerase and initiating transcription of a downstream (3′ direction) coding or non-coding sequence. For purposes of defining the present invention, the promoter sequence is bounded at its 3′ terminus by the transcription initiation site and extends upstream (5′ direction) to include the minimum number of bases or elements necessary to initiate transcription at levels detectable above background. Within the promoter sequence will be found a transcription initiation site, as well as protein binding domains responsible for the binding of RNA polymerase. Eukaryotic promoters will often, but not always, contain “TATA” boxes and “CAT” boxes. Various promoters, including inducible promoters, may be used to drive the various vectors of the present invention.

A promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein.), it may be a spatially restricted promoter (i.e., transcriptional control element, enhancer, etc.)(e.g., tissue specific promoter, cell type specific promoter, etc.), and it may be a temporally restricted promoter (i.e., the promoter is in the “ON” state or “OFF” state during specific stages of embryonic development or during specific stages of a biological process, e.g., hair follicle cycle in mice).

The term “naturally-occurring” or “unmodified” as used herein as applied to a nucleic acid, a polypeptide, a cell, or an organism, refers to a nucleic acid, polypeptide, cell, or organism that is found in nature. For example, a polypeptide or polynucleotide sequence that is present in an organism (including viruses) that can be isolated from a source in nature is naturally occurring.

“Heterologous,” as used herein, means a nucleotide or polypeptide sequence that is not found in the native nucleic acid or protein, respectively. A heterologous nucleic acid sequence may be linked to a naturally-occurring nucleic acid sequence (or a variant thereof) (e.g., by genetic engineering) to generate a chimeric nucleotide sequence encoding a chimeric polypeptide.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “DNA regulatory sequences”, below). The term “recombinant” nucleic acid refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a codon encoding the same amino acid, a conservative amino acid, or a non-conservative amino acid.

A “vector” or “expression vector” is a replicon, such as plasmid, phage, virus, or cosmid, to which another DNA segment, i.e. an “insert”, may be attached so as to bring about the replication of the attached segment in a cell.

An “expression cassette” comprises a DNA coding sequence operably linked to a promoter.

“Operably linked” refers to a juxtaposition wherein the components so described are in a relationship permitting them to function in their intended manner. For instance, a promoter is operably linked to a coding sequence if the promoter affects its transcription or expression. Likewise, the coding sequence can be said to be operably linked to the promoter.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

“Nuclease” and “endonuclease” (e.g., DNA nuclease and/or DNA endonuclease) are used interchangeably herein to mean an enzyme which possesses catalytic activity for DNA cleavage.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom, and includes: (a) preventing the disease or symptom from occurring in a subject which may be predisposed to acquiring the disease or symptom but has not yet been diagnosed as having it; (b) inhibiting the disease or symptom, i.e., arresting its development; or (c) relieving the disease, i.e., causing regression of the disease. The therapeutic agent may be administered before, during or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any subject for whom diagnosis, treatment, or therapy is desired (e.g., mammal, pet, farm animal, horse, pig, cow, donkey, rat, mouse, non-human primate, human).

General methods in molecular and cellular biochemistry can be found in such standard textbooks as Molecular Cloning: A Laboratory Manual, 3rd Ed. (Sambrook et al., HaRBor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

### DETAILED DESCRIPTION

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges, and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs.

Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It is noted that as used herein and in the appended claims, the singular forms “a,” “an,” and “the” include plural referents unless the context clearly dictates otherwise. Thus, for example, reference to “a cell” includes a plurality of such cells and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Methods and compositions are provided for facilitating efficient adeno-associated virus (AAV)-based homologous recombination (HR). Aspects of the methods include contacting a cell (e.g., a population of cells) with a ribonucleotide reductase inhibitor, which provides for increased HR efficiency compared to performing HR in the absence of the inhibitor. The cell is also contacted with a recombinant adeno-associated virus (rAAV) that includes a donor DNA having a sequence cassette (i.e., a nucleotide sequence of interest) flanked by homology arms that facilitate integration of the sequence cassette into a target genomic locus (via HR)—for example the homology arms are homologous to sequences flanking an integration site in the targeted genomic locus. The sequence cassette includes a transgene sequence (e.g., a sequence that encodes a protein of interest such as a therapeutic protein, a non-coding RNA such as an siRNA, and the like). Thus, the subject methods provide for efficient integration of the sequence cassette, and therefore the transgene sequence, into a genomic locus via AAV delivery and HR Reagents, compositions, and kits/systems that find use in practicing the subject methods are also provided. These and other objects, advantages, and features of the invention will become apparent to those persons skilled in the art upon reading the details of the compositions and methods as more fully described below.

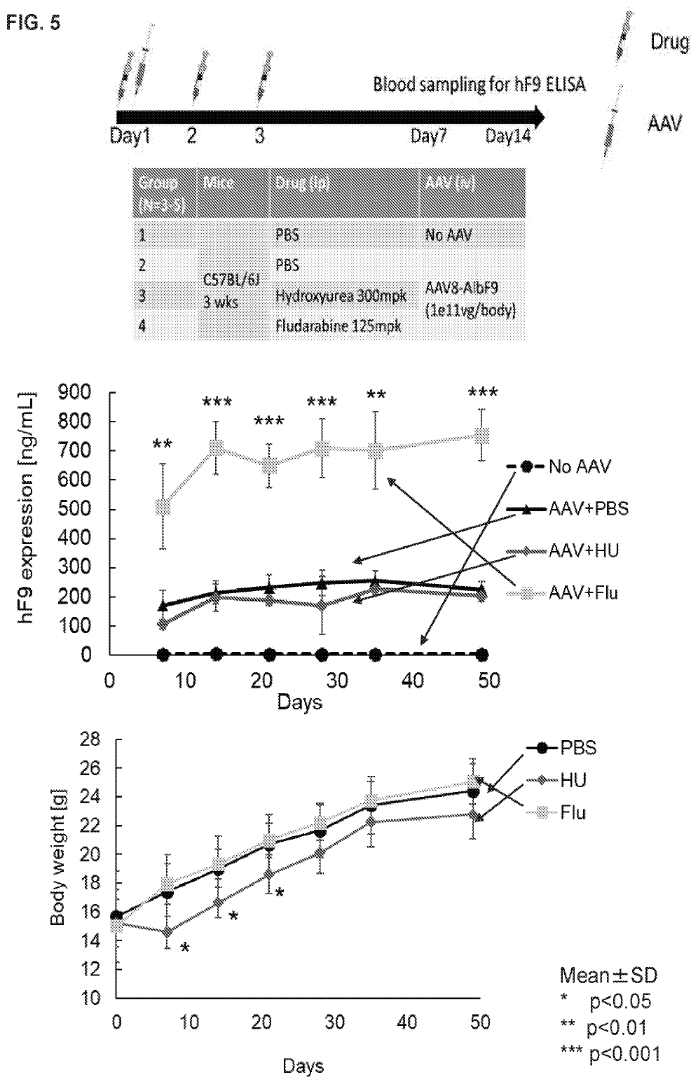

For example, the methods and compositions provided by the present disclosure provide for increased AAV-based HR efficiency due to contacting a cell (e.g., a population of cells) with a ribonucleotide reductase (RNR) inhibitor. As such, the methods and compositions provided by the present disclosure provide for increased AAV-based HR efficiency compared to control (e.g., the HR efficiency when the cells are not contacted with the RNR inhibitor). In some cases contact with an RNR inhibitor provides for a 1.2-fold or more increase in efficiency (meaning that the HR efficiency with RNR inhibitor contact is 1.2-fold or more the efficiency without RNR inhibitor contact—which is a 20% increase or more). In some cases contact with an RNR inhibitor provides for a 2-fold or more increase in efficiency (which is a 100% increase). In some cases contact with an RNR inhibitor provides for a 1.2-fold or more increase in efficiency (1.5-fold or more, 1.8-fold or more, 2-fold or more, 2.5-fold or more, 3-fold or more, 4-fold or more, or 5-fold or more increase in efficiency). As an illustrative example, treatment with hydroxyurea (HU) or Fludarabine (Flu) in FIG. 4 resulted in—3.5-fold increase in HR efficiency.

### RNR Inhibitors

Ribonucleotide reductase (RNR) inhibitors are compounds that inhibit/block the activity of RNR. RNR is a multi-subunit enzyme that converts ribonucleotides into deoxyribonucleotides. A pool of available deoxyribonucleotides is important for DNA replication during S phase of the cell cycle as well as multiple DNA repair processes. In humans, the RNR1 subunit is encoded by the RRM1 gene while there are two isoforms of the RNR2 subunit, encoded by the RRM2 and RRM2B genes.

Examples of known RNR inhibitors include but are not limited to: hydroxyurea (HU); Motexafin gadolinium (an inhibitor of thioredoxin reductase and ribonucleotide reductase); Fludarabine (a purine analog which inhibits DNA synthesis by interfering with ribonucleotide reductase and DNA polymerase—S phase specific); Cladribine (a purine analog which inhibits DNA synthesis); Gemcitabine (a cystydin analog which inhibits DNA synthesis); Tezacitabine (a purine nucleoside analogue which inhibits DNA synthesis); Triapine (or 3-AP) (inhibits both RRM2/p53R2 and also an iron chelator); and Gallium maltolate (a mimetic of Fe3+ which is essential for ribonucleotide reductase). The IUPAC names for the above listed compounds are as follows:

Hydroxyurea (HU)
hydroxyurea

Motexafin gadolinium (Gadolinium texaphyrin)
acetic acid; 344,5-diethyl-24-(3-hydroxypropyl)-16,17-bis[2-[2-(2-methoxyethoxy)ethoxy]ethoxy]-10,23-dimethyl-13,20,25,26-tetraza-27-azanidapentacyclo[20.2.1.13,6.18,11.014,19]heptacosa-1(25),2,4,6,8(26),9,11,13,15,17,19,21,23-tridecaen-9-yl]propan-1-ol; gadolinium

Fludarabine
(2R,3S,4S,5R)-2-(6-amino-2-fluoropurin-9-yl)-5-(hydroxymethyl)oxolane-3,4-diol

Cladribine
(2R,3S,5R)-5-(6-amino-2-chloropurin-9-yl)-2-(hydroxymethyl)oxolan-3-ol

Gemcitabine
4-amino-1-R2R,4R,5R)-3,3-difluoro-4-hydroxy-5-(hydroxymethyl)oxolan-2-yl]pyrimidin-2-one

Tezacitabine (2′-Deoxy-2′-(fluoromethylene)cytidine)
4-amino-1-[(2R,3E,4S,5R)-3-(fluoromethylidene)-4-hydroxy-5-(hydroxymethyl)oxolan-2-yl]pyrimidin-2-one

Triapine
[(E)-(3-aminopyridin-2-yl)methylideneamino]thiourea

Gallium maltolate
gallium; 2-methyl-4-oxopyran-3-olate

In some cases a subject RNR inhibitor includes one or more of the above compounds—e.g., one or more compounds selected from: HU, Gemcitabine, Fludarabine, Motexafin gadolinium, Cladribine, Tezacitabine, and Gallium maltolate. For example in some cases a subject RNR inhibitor includes HU, Gemcitabine, Fludarabine, Motexafin gadolinium, Cladribine, Tezacitabine, Gallium maltolate, or any combination thereof. In other words, in some cases a subject RNR inhibitor includes one or more of: HU, Gemcitabine, Fludarabine, Motexafin gadolinium, Cladribine, Tezacitabine, and Gallium maltolate. In some cases a subject RNR inhibitor includes one or more of: HU, Fludarabine, and Gemcitabine. In some cases a subject RNR inhibitor includes Fludarabine. In some cases a subject RNR inhibitor includes Gemcitabine. In some cases a subject RNR inhibitor includes HU.

RNR inhibition can also be achieved via targeting by non-coding RNAs (e.g., antisense RNAs and siRNAs). For example, an siRNA targeting one or more subunits of the RNR enzyme can be an RNR inhibitor. Thus, in some cases a subject RNR inhibitor includes a non-coding RNA (e.g., an siRNA) that targets RNR.

A target cell (e.g., a population of cells) can be in vitro, ex vivo, or in vivo. For example, the target cell can be a cell in culture or can be a cell in vivo (e.g., the cell can be contacted with the RNR inhibitor by administering the RNR inhibitor to an individual).

In some cases a target cell (e.g., a population of cells) is contacted with the ribonucleotide reductase inhibitor and the targeting vector (rAAV) at the same time (e.g., both are delivered as part of the same formulation or are delivered at the time in separate formulations). In some cases a target cell (e.g., a population of cells) is contacted with the ribonucleotide reductase inhibitor prior to contact with the ribonucleotide reductase inhibitor. In some cases a target cell (e.g., a population of cells) is contacted with the ribonucleotide reductase inhibitor for a period of time in a range of from 1-20 hours (e.g., from 1-18 hours, 1-16 hours, 1-14 hours, 1-12 hours, 1-10 hours, 1-8 hours, 1-6 hours, 1-4 hours, 2-20 hours, 2-18 hours, 2-16 hours, 2-14 hours, 2-12 hours, 2-10 hours, 2-8 hours, 2-6 hours, 2-4 hours, 4-20 hours, 4-18 hours, 4-16 hours, 4-14 hours, 4-12 hours, 4-10 hours, 4-8 hours, 4-6 hours, 6-20 hours, 6-18 hours, 6-16 hours, 6-14 hours, 6-12 hours, 6-10 hours, 6-8 hours, 10-20 hours, 10-18 hours, 10-16 hours, 10-14 hours, or 10-12 hours). In some cases a target cell (e.g., a population of cells) is contacted with the ribonucleotide reductase inhibitor for 30 or more minutes (e.g., 1 or more, 2 or more, 3 or more, 5 or more, 8 or more, 10 or more, 12 or more, 14 or more, 16 or more, or 18 or more hours). In some cases a target cell (e.g., a population of cells) is contacted with the ribonucleotide reductase inhibitor for a period of time in a range of from 1-20 hours (e.g., from 1-18 hours, 1-16 hours, 1-14 hours, 1-12 hours, 1-10 hours, 1-8 hours, 1-6 hours, 1-4 hours, 2-20 hours, 2-18 hours, 2-16 hours, 2-14 hours, 2-12 hours, 2-10 hours, 2-8 hours, 2-6 hours, 2-4 hours, 4-20 hours, 4-18 hours, 4-16 hours, 4-14 hours, 4-12 hours, 4-10 hours, 4-8 hours, 4-6 hours, 6-20 hours, 6-18 hours, 6-16 hours, 6-14 hours, 6-12 hours, 6-10 hours, 6-8 hours, 10-20 hours, 10-18 hours, 10-16 hours, 10-14 hours, or 10-12 hours) and is then contacted with the targeting vector. In some cases a target cell (e.g., a population of cells) is contacted with the ribonucleotide reductase inhibitor for 30 or more minutes (e.g., 1 or more, 2 or more, 3 or more, 5 or more, 8 or more, 10 or more, 12 or more, 14 or more, 16 or more, or 18 or more hours) and is then contacted with the targeting vector.

As would be understood by one of ordinary skill in the art, any convenient dose/concentration of an RNR inhibitor can be used. For example, in some cases (e.g., in some cases in which the contacted cell population is in vitro or ex vivo) fludarabine is used at a concentration in a range of from 20 μM to 500 μM (e.g., from 20 μM to 400 μM, 20 μM to 300 μM, 20 μM to 200 μM, 20 μM to 150 μM, 50 μM to 500 μM, 50 μM to 400 μM, 50 μM to 300 μM, 50 μM to 200 μM, 50 μM to 150 μM, or 75 μM to 125 μM). In some cases (e.g., in some cases in which the contacted cell population is in vitro or ex vivo) hydroxyurea is used at a concentration in a range of from 0.5 mM to 50 mM (e.g., from 0.5 mM to 40 mM, 0.5 mM to 30 mM, 0.5 mM to 25 mM, 0.5 mM to 20 mM, 0.5 mM to 15 mM, 0.5 mM to 10 mM, 1 mM to 50 mM, 1 mM to 40 mM, 1 mM to 30 mM, 1 mM to 25 mM, 1 mM to 20 mM, 1 mM to 15 mM, 1 mM to 10 mM, 2 mM to 50 mM, 2 mM to 40 mM, 2 mM to 30 mM, 2 mM to 25 mM, 2 mM to 20 mM, 2 mM to 15 mM, 2 mM to 10 mM, 2 mM to 8 mM, 2.5 mM to 7.5 mM, or 5 mM to 15 mM). In some cases (e.g., in some cases in which the contacted cell population is in vitro or ex vivo) Gemcitabine is used at a concentration in a range of from 5 nM to 500 nM (e.g., from 5 nM to 400 nM, 5 nM to 300 nM, 5 nM to 200 nM, 5 nM to 100 nM, 10 nM to 500 nM, 10 nM to 400 nM, 10 nM to 300 nM, 10 nM to 200 nM, 10 nM to 100 nM, 20 nM to 500 nM, 20 nM to 400 nM, 20 nM to 300 nM, 20 nM to 200 nM, 20 nM to 100 nM, 40 nM to 500 nM, 40 nM to 400 nM, 40 nM to 300 nM, 40 nM to 200 nM, 40 nM to 100 nM, 50 nM to 500 nM, 50 nM to 400 nM, 50 nM to 300 nM, 50 nM to 200 nM, 50 nM to 100 nM, 60 nM to 500 nM, 60 nM to 400 nM, 60 nM to 300 nM, 60 nM to 200 nM, or 60 nM to 100 nM).

In some cases an RNR inhibitor is administer to an individual (e.g., a population of cells to be contacted is in vivo in the individual). For example, in some cases fludarabine is administered to an individual at a dose in a range of from 0.5 to 200 milligrams per kilogram body weight (mpk) (e.g., 0.5 to 150 mpk, 0.5 to 125 mpk, 0.5 to 100 mpk, 0.5 to 80 mpk, 0.5 to 70 mpk, 0.5 to 60 mpk, 0.5 to 55 mpk, 0.5 to 50 mpk, 0.5 to 45 mpk, 0.5 to 40 mpk, 1 to 200 mpk, 1 to 150 mpk, 1 to 125 mpk, 1 to 100 mpk, 1 to 80 mpk, 1 to 70 mpk, 1 to 60 mpk, 1 to 55 mpk, 1 to 50 mpk, 1 to 45 mpk, 1 to 40 mpk, 2 to 200 mpk, 2 to 150 mpk, 2 to 125 mpk, 2 to 100 mpk, 2 to 80 mpk, 2 to 70 mpk, 2 to 60 mpk, 2 to 55 mpk, 2 to 50 mpk, 2 to 45 mpk, 2 to 40 mpk, 5 to 200 mpk, 5 to 150 mpk, 5 to 125 mpk, 5 to 100 mpk, 5 to 80 mpk, 5 to 70 mpk, 5 to 60 mpk, 5 to 55 mpk, 5 to 50 mpk, 5 to 45 mpk, 5 to 40 mpk, 10 to 200 mpk, 10 to 150 mpk, 10 to 125 mpk, 10 to 100 mpk, 10 to 80 mpk, 10 to 70 mpk, 10 to 60 mpk, 10 to 55 mpk, 10 to 50 mpk, 10 to 45 mpk, 10 to 40 mpk, 20 to 200 mpk, 20 to 150 mpk, 20 to 125 mpk, 20 to 100 mpk, 20 to 80 mpk, 20 to 70 mpk, 20 to 60 mpk, 20 to 55 mpk, 20 to 50 mpk, 20 to 45 mpk, or 20 to 40 mpk).

Cells may be contacted with the subject RNR inhibitors and targeting vectors in vitro or in vivo. If contacted in vitro, cells may be from established cell lines or they may be primary cells, where “primary cells”, “primary cell lines”, and “primary cultures” are used interchangeably herein to refer to cells and cells cultures that have been derived from a subject and either modified without significant additional culturing, i.e. modified “ex vivo”, e.g. for return to the subject, or allowed to grow in vitro for a limited number of passages, i.e. splittings, of the culture. For example, primary cultures are cultures that may have been passaged 0 times, 1 time, 2 times, 4 times, 5 times, 10 times, or 15 times, but not enough times go through the crisis stage. Typically, the primary cell lines of the present invention are maintained for fewer than 10 passages in vitro. Typically, the cells to be contacted are permissive of homologous recombination.

If the cells are primary cells, they may be harvest from an individual by any convenient method. For example, leukocytes may be conveniently harvested by apheresis, leukocytapheresis, density gradient separation, etc., while cells from tissues such as skin, muscle, bone marrow, spleen, liver, pancreas, lung, intestine, stomach, etc. are most conveniently harvested by biopsy. An appropriate solution may be used for dispersion or suspension of the harvested cells. Such solution will generally be a balanced salt solution, e.g. normal saline, PBS, Hank's balanced salt solution, etc., conveniently supplemented with fetal calf serum or other naturally occurring factors, in conjunction with an acceptable buffer at low concentration, generally from 5-25 mM. Convenient buffers include HEPES, phosphate buffers, lactate buffers, etc. The cells may be used immediately, or they may be stored, frozen, for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

In some embodiments, the sequence cassette integrates into two chromosomes (e.g., maternal and paternal) of the target cell such that the integration is homozygotic.

### AAV Vector

In practicing the subject methods, the transgene sequence to be integrated into the genome of the cell is provided to cells on a vector, referred to herein as a “targeting vector”. In other words, cells are contacted with a targeting vector that comprises a donor DNA, which includes a transgene sequence to be integrated into the cellular genome by targeted integration. As such, in practicing the subject methods, a cell (e.g. a mitotic cell, a post-mitotic cell) is contacted in vitro or in vivo with a targeting vector such that the targeting vector is taken up by the cells. Methods and systems for packaging nucleic acid vectors into viral capsids, harvesting the viral particles comprising the nucleic acid vector, and contacting cells with the viral particles comprising the nucleic acid vector are also well known in the art, any of which may be used. The targeting vector will include a donor DNA having a nucleotide sequence cassette (a nucleotide sequence of interest) flanked by homology arms (sequences of homology to the target integration site), e.g., as heterologous sequences in association with viral genomic sequence, e.g. inverted terminal repeats (ITRs). The nucleotide sequence cassette will include a transgene sequence. In some cases a subject sequence cassette will also include, 5′ or 3′ of the transgene sequence, a nucleic acid sequence that promotes the production of two independent gene products.

By adeno-associated virus, or “AAV” it is meant the virus itself or derivatives thereof. The term covers all subtypes and both naturally occurring and recombinant forms, except where required otherwise, for example, AAV type 1 (AAV-1), AAV type 2 (AAV-2), AAV type 3 (AAV-3), AAV type 4 (AAV-4), AAV type 5 (AAV-5), AAV type 6 (AAV-6), AAV type 7 (AAV-7), AAV type 8 (AAV-8), AAV type 9 (AAV-9), AAV type 10 (AAV-10), AAV type 11 (AAV-11), avian AAV, bovine AAV, canine AAV, equine AAV, primate AAV, non-primate AAV, ovine AAV, a hybrid AAV (i.e., an AAV comprising a capsid protein of one AAV subtype and genomic material of another subtype), an AAV comprising a mutant AAV capsid protein or a chimeric AAV capsid (i.e. a capsid protein with regions or domains or individual amino acids that are derived from two or more different serotypes of AAV, e.g. AAV-DJ, AAV-LK3, AAV-LK19). “Primate AAV” refers to AAV that infect primates, “non-primate AAV” refers to AAV that infect non-primate mammals, “bovine AAV” refers to AAV that infect bovine mammals, etc.

By a “recombinant AAV vector”, or “rAAV vector” it is meant an AAV virus or AAV viral chromosomal material comprising a polynucleotide sequence not of AAV origin (i.e., a polynucleotide heterologous to AAV), typically a nucleic acid sequence of interest to be integrated into the cell following the subject methods. In general, the heterologous polynucleotide is flanked by at least one, and generally by two AAV inverted terminal repeat sequences (ITRs). In some instances, the recombinant viral vector also comprises viral genes important for the packaging of the recombinant viral vector material. By “packaging” it is meant a series of intracellular events that result in the assembly and encapsidation of a viral particle, e.g. an AAV viral particle. Examples of nucleic acid sequences important for AAV packaging (i.e., “packaging genes”) include the AAV “rep” and “cap” genes, which encode for replication and encapsidation proteins of adeno-associated virus, respectively. The term rAAV vector encompasses both rAAV vector particles and rAAV vector plasmids.

A “viral particle” refers to a single unit of virus comprising a capsid encapsidating a virus-based polynucleotide, e.g. the viral genome (as in a wild type virus), or, e.g., the subject targeting vector (as in a recombinant virus). An “AAV viral particle” refers to a viral particle composed of at least one AAV capsid protein (typically by all of the capsid proteins of a wild-type AAV) and an encapsidated polynucleotide AAV vector. If the particle comprises a heterologous polynucleotide (i.e. a polynucleotide other than a wild-type AAV genome, such as a transgene to be delivered to a mammalian cell), it is typically referred to as an “rAAV vector particle” or simply an “rAAV vector”. Thus, production of rAAV particle necessarily includes production of rAAV vector, as such a vector is contained within an rAAV particle.

A rAAV virion can be constructed using methods that are well known in the art. See, e.g., Koerber et al. (2009) Mol. Ther. 17:2088; Koerber et al. (2008) Mol Ther. 16:1703-1709; U.S. Pat. Nos. 7,439,065, 6,951,758, and 6,491,907. For example, the heterologous sequence(s) can be directly inserted into an AAV genome which has had the major AAV open reading frames (“ORFs”) excised therefrom. Other portions of the AAV genome can also be deleted, so long as a sufficient portion of the ITRs remain to allow for replication and packaging functions. Such constructs can be designed using techniques well known in the art. See, e.g., U.S. Pat. Nos. 5,173,414 and 5,139,941; International Publication Nos. WO 92/01070 (published Jan. 23, 1992) and WO 93/03769 (published Mar. 4, 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Curr. Topics Microbiol. Immunol. 158:97-129; Kotin, R. M. (1994) Human Gene Therapy 5:793-801; Shelling and Smith (1994) Gene Therapy 1:165-169; and Zhou et al. (1994) J. Exp. Med. 179:1867-1875.

In order to produce rAAV virions, an AAV expression vector is introduced into a suitable host cell using known techniques, such as by transfection. A number of transfection techniques are generally known in the art. See, e.g., Graham et al. (1973) Virology, 52:456, Sambrook et al. (1989) Molecular Cloning, A Laboratory Manual, Cold Spring Harbor Laboratories, New York, Davis et al. (1986) Basic Methods in Molecular Biology, Elsevier, and Chu et al. (1981) Gene 13:197, Particularly suitable transfection methods include calcium phosphate co-precipitation (Graham et al. (1973) Virol. 52:456-467), direct micro-injection into cultured cells (Capecchi, M. R. (1980) Cell 22:479-488), electroporation (Shigekawa et al. (1988) BioTechnigues 6:742-751), liposome mediated gene transfer (Mannino et al. (1988) BioTechniques 6:682-690), lipid-mediated transduction (Felgner et al. (1987) Proc. Natl. Acad. Sci. USA 84:7413-7417), and nucleic acid delivery using high-velocity microprojectiles (Klein et al. (1987) Nature 327:70-73).

Suitable host cells for producing rAAV virions include microorganisms, yeast cells, insect cells, and mammalian cells, that can be, or have been, used as recipients of a heterologous DNA molecule. The term includes the progeny of the original cell which has been transfected. Thus, a “host cell” as used herein generally refers to a cell which has been transfected with an exogenous DNA sequence. Cells from the stable human cell line, 293 (readily available through, e.g., the American Type Culture Collection under Accession Number ATCC CRL1573) can be used. For example, the human cell line 293 is a human embryonic kidney cell line that has been transformed with adenovirus type-5 DNA fragments (Graham et al. (1977) J. Gen. Virol. 36:59), and expresses the adenoviral E1a and E1b genes (Aiello et al. (1979) Virology 94:460). The 293 cell line is readily transfected, and provides a convenient platform in which to produce rAAV virions. Methods of producing an AAV virion in insect cells are known in the art, and can be used to produce a subject rAAV virion. See, e.g., U.S. Patent Publication No. 2009/0203071; U.S. Pat. No. 7,271,002; and Chen (2008) Mol. Ther. 16:924.

AAV virus that is produced may be replication competent or replication-incompetent. A “replication-competent” virus (e.g. a replication-competent AAV) refers to a phenotypically wild-type virus that is infectious, and is also capable of being replicated in an infected cell (e.g., in the presence of a helper virus or helper virus functions). In the case of AAV, replication competence generally requires the presence of functional AAV packaging genes. In general, rAAV vectors as described herein are replication-incompetent in mammalian cells (especially in human cells) by virtue of the lack of one or more AAV packaging genes. Typically, such rAAV vectors lack any AAV packaging gene sequences in order to minimize the possibility that replication competent AAV are generated by recombination between AAV packaging genes and an incoming rAAV vector. In many embodiments, rAAV vector preparations as described herein are those which contain few if any replication competent AAV (rcAAV, also referred to as RCA) (e.g., less than about 1 rcAAV per 102 rAAV particles, less than about 1 rcAAV per 104 rAAV particles, less than about 1 rcAAV per 108 rAAV particles, less than about 1 rcAAV per 1012 rAAV particles, or no rcAAV).

To induced DNA integration in vitro, the targeting vector (rAAV) can be provided to the cells for about 30 minutes to about 24 hours, e.g., 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The targeting vector may be provided to the subject cells one or more times, e.g. one time, twice, three times, or more than three times, and the cells allowed to incubate with the target vector for some amount of time following each contacting event e.g. 16-24 hours, after which time the media is replaced with fresh media and the cells are cultured further.

Contacting the cells with the targeting vector may occur in any culture media and under any culture conditions that promote the survival of the cells. For example, cells may be suspended in any appropriate nutrient medium that is convenient, such as Iscove's modified DMEM or RPMI 1640, supplemented with fetal calf serum or heat inactivated goat serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Typically, an effective amount of targeting vector is provided to the cells to promote recombination and integration. An effective amount of target vector is the amount to induce an increase in the number of cells in which integration of the transgene is observed relative to a negative control, e.g. a cell contacted with an empty vector. The amount of integration may be measured by any convenient method. For example, the presence of the gene of interest in the locus may be detected by, e.g., flow cytometry. PCR or Southern hybridization may be performed using primers that will amplify the target locus to detect the presence of the insertion. The expression or activity of the integrated gene of interest may be determined by Western, ELISA, testing for protein activity, etc. e.g. 2 hours, 4 hours, 8 hours, 12 hours, 24 hours, 36 hours, 48 hours, 72 hours or more after contact with the donor polynucleotide. As another example, integration may be measured by co-integrating an imaging marker or a selectable marker, and detecting the presence of the imaging or selectable marker in the cells.

In some cases genetic modification of the cell using the subject compositions and methods will not be accompanied by disruption of the expression of the gene at the modified locus, i.e. the target locus. In other words, the normal expression of the gene at the target locus is maintained spatially, temporally, and at levels that are substantially unchanged from normal levels, for example, at levels that differ 5-fold or less from normal levels, e.g. 4-fold or less, or 3-fold or less, more usually 2-fold or less from normal levels, following targeted integration of the gene of interest into the target locus.

In some instances, the population of cells may be enriched for those comprising the transgene by separating the genetically modified cells from the remaining population. Separation of genetically modified cells typically relies upon the expression of a selectable marker that is co-integrated into the target locus. By a “selectable marker” it is meant an agent that can be used to select cells, e.g. cells that have been targeted by compositions of the subject application. In some instances, the selection may be positive selection; that is, the cells are isolated from a population, e.g. to create an enriched population of cells comprising the genetic modification. In other instances, the selection may be negative selection; that is, the population is isolated away from the cells, e.g. to create an enriched population of cells that do not comprise the genetic modification. Separation may be by any convenient separation technique appropriate for the selectable marker used. For example, if a fluorescent marker has been inserted, cells may be separated by fluorescence activated cell sorting, whereas if a cell surface marker has been inserted, cells may be separated from the heterogeneous population by affinity separation techniques, e.g. magnetic separation, affinity chromatography, “panning” with an affinity reagent attached to a solid matrix, or other convenient technique. Techniques providing accurate separation include fluorescence activated cell sorters, which can have varying degrees of sophistication, such as multiple color channels, low angle and obtuse light scattering detecting channels, impedance channels, etc. The cells may be selected against dead cells by employing dyes associated with dead cells (e.g. propidium iodide). Any technique may be employed which is not unduly detrimental to the viability of the genetically modified cells.

Cell compositions that are highly enriched for cells comprising the transgene are achieved in this manner. By “highly enriched”, it is meant that the genetically modified cells will be 70% or more, 75% or more, 80% or more, 85% or more, 90% or more of the cell composition, for example, about 95% or more, or 98% or more of the cell composition. In other words, the composition may be a substantially pure composition of genetically modified cells.

Genetically modified cells produced by the methods described herein may be used immediately. Alternatively, the cells may be frozen at liquid nitrogen temperatures and stored for long periods of time, being thawed and capable of being reused. In such cases, the cells will usually be frozen in 10% DMSO, 50% serum, 40% buffered medium, or some other such solution as is commonly used in the art to preserve cells at such freezing temperatures, and thawed in a manner as commonly known in the art for thawing frozen cultured cells.

The genetically modified cells may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

Cells that have been genetically modified in this way may be transplanted to a subject for purposes such as gene therapy, e.g. to treat a disease or as an antiviral, antipathogenic, or anticancer therapeutic, for the production of genetically modified organisms in agriculture, or for biological research. The subject may be a neonate, a juvenile, or an adult. Of particular interest are mammalian subjects Mammalian species that may be treated with the present methods include canines and felines; equines; bovines; ovines; etc. and primates, particularly humans.

Animal models, particularly small mammals, e.g. murine, lagomorpha, etc. may be used for experimental investigations.

Cells may be provided to the subject alone or with a suitable substrate or matrix, e.g. to support their growth and/or organization in the tissue to which they are being transplanted. Usually, at least 1×10³ cells will be administered, for example 5×10³ cells, 1×10⁴ cells, 5×10⁴ cells, 1×10⁵ cells, 1×10⁶ cells or more. The cells may be introduced to the subject via any of the following routes: parenteral, subcutaneous, intravenous, intracranial, intraspinal, intraocular, or into spinal fluid. The cells may be introduced by injection, catheter, or the like. Examples of methods for local delivery, that is, delivery to the site of injury, include, e.g. through an Ommaya reservoir, e.g. for intrathecal delivery (see e.g. U.S. Pat. Nos. 5,222,982 and 5,385,582, incorporated herein by reference); by bolus injection, e.g. by a syringe, e.g. into a joint; by continuous infusion, e.g. by cannulation, e.g. with convection (see e.g. US Application No. 20070254842, incorporated here by reference); or by implanting a device upon which the cells have been reversably affixed (see e.g. US Application Nos. 20080081064 and 20090196903, incorporated herein by reference)

The number of administrations of treatment to a subject may vary. Introducing the genetically modified cells into the subject may be a one-time event; but in certain situations, such treatment may elicit improvement for a limited period of time and require an on-going series of repeated treatments. In other situations, multiple administrations of the genetically modified cells may be required before an effect is observed. The exact protocols depend upon the disease or condition, the stage of the disease and parameters of the individual subject being treated.

In some aspects, the RNR inhibitor and targeting vector (rAAV) are employed to modify cellular

DNA in vivo. In these in vivo embodiments, the RNR inhibitor and rAAV may be administered by any of a number of well-known methods in the art for the administration of compounds and nucleic acids to a subject. The RNR inhibitor and rAAV can be incorporated into a variety of formulations. More particularly, the RNR inhibitor and rAAV can be formulated into pharmaceutical compositions by combination with appropriate pharmaceutically acceptable carriers or diluents—either together as part of the same formulation or as two separate formulations.

Pharmaceutical preparations are compositions that include an RNR inhibitor and/or an rAAV present in a pharmaceutically acceptable vehicle. “Pharmaceutically acceptable vehicles” may be vehicles approved by a regulatory agency of the Federal or a state government or listed in the U.S. Pharmacopeia or other generally recognized pharmacopeia for use in mammals, such as humans. The term “vehicle” refers to a diluent, adjuvant, excipient, or carrier with which a compound of the disclosure is formulated for administration to a mammal. Such pharmaceutical vehicles can be lipids, e.g. liposomes, e.g. liposome dendrimers; liquids, such as water and oils, including those of petroleum, animal, vegetable or synthetic origin, such as peanut oil, soybean oil, mineral oil, sesame oil and the like, saline; gum acacia, gelatin, starch paste, talc, keratin, colloidal silica, urea, and the like. In addition, auxiliary, stabilizing, thickening, lubricating and coloring agents may be used. Pharmaceutical compositions may be formulated into preparations in solid, semi-solid, liquid or gaseous forms, such as tablets, capsules, powders, granules, ointments, solutions, suppositories, injections, inhalants, gels, microspheres, and aerosols.

As such, administration of the an RNR inhibitor and/or an rAAV can be achieved in various ways, including systemically or locally. For example, administration of the an RNR inhibitor and/or an rAAV can be achieved in various ways including (but not limited to): injection (e.g., local injection or intravascular injection), intramuscular (im), oral, buccal, rectal, parenteral, intraperitoneal (ip), intravascular (iv), subcutaneous (sc), intraocular, intradermal, transdermal, intracheal, etc., administration. The active agent may be systemic after administration or may be localized by the use of regional administration, intramural administration, or use of an implant that acts to retain the active dose at the site of implantation. The active agent may be formulated for immediate activity or it may be formulated for sustained release.

In some cases an RNR inhibitor and/or an rAAV is administered to an individual at least once a day for two or more consecutive days. In some cases an RNR inhibitor and/or an rAAV is administered once daily administration for 5 consecutive days every 28 days.

In some cases, a subject RNR inhibitor (e.g., hydroxyurea (HU), motexafin gadolinium, fludarabine, cladribine, gemcitabine, tezacitabine, triapine, gallium maltolate, and the like) is administered within about 30 days (e.g., within about 25 days, 21 days, 17 days, 14 days, 10 days, or 7 days) of administration of a donor nucleic acid (e.g., an rAAV) to an individual. For example, in some cases fludarabine is administered within about 30 days (e.g., within about 25 days, 21 days, 17 days, 14 days, 10 days, or 7 days) of administration of a donor nucleic acid (e.g., an rAAV) to an individual.

In some cases, a subject RNR inhibitor (e.g., hydroxyurea (HU), motexafin gadolinium, fludarabine, cladribine, gemcitabine, tezacitabine, triapine, gallium maltolate, and the like) is administered within about 25 days (g., within about 21 days, 17 days, 14 days, 10 days, or 7 days) of administration of a donor nucleic acid (e.g., an rAAV) to an individual. For example, in some cases fludarabine is administered within about 25 days (g., within about 21 days, 17 days, 14 days, 10 days, or 7 days) of administration of a donor nucleic acid (e.g., an rAAV) to an individual.

In some cases, a subject RNR inhibitor (e.g., hydroxyurea (HU), motexafin gadolinium, fludarabine, cladribine, gemcitabine, tezacitabine, triapine, gallium maltolate, and the like) is administered within about 21 days (e.g., within about 17 days, 14 days, 10 days, or 7 days) of administration of a donor nucleic acid (e.g., an rAAV) to an individual. For example, in some cases fludarabine is administered within about 21 days (e.g., within about 17 days, 14 days, 10 days, or 7 days) of administration of a donor nucleic acid (e.g., an rAAV) to an individual.

In some cases, a subject RNR inhibitor (e.g., hydroxyurea (HU), motexafin gadolinium, fludarabine, cladribine, gemcitabine, tezacitabine, triapine, gallium maltolate, and the like) is administered in a range of from about 3 days to about 30 days (e.g., in a range of from 3 days to 25 days, 3 days to 21 days, 7 days to 30 days, 7 days to 25 days, 7 days to 21 days, 10 days to 30 days, 10 days to 25 days, 10 days to 21 days, 14 days to 30 days, 14 days to 25 days, or 14 days to 21 days) after administration of a donor nucleic acid (e.g., an rAAV) to an individual. For example, in some cases fludarabine is administered in a range of from about 3 days to about 30 days (e.g., in a range of from 3 days to 25 days, 3 days to 21 days, 7 days to 30 days, 7 days to 25 days, 7 days to 21 days, 10 days to 30 days, 10 days to 25 days, 10 days to 21 days, 14 days to 30 days, 14 days to 25 days, or 14 days to 21 days) after administration of a donor nucleic acid (e.g., an rAAV) to an individual.

In some cases, a subject RNR inhibitor (e.g., hydroxyurea (HU), motexafin gadolinium, fludarabine, cladribine, gemcitabine, tezacitabine, triapine, gallium maltolate, and the like) is administered in a range of from about 10 days to about 30 days (e.g., in a range of from 10 days to 25 days, 10 days to 21 days, 14 days to 30 days, 14 days to 25 days, or 14 days to 21 days) after administration of a donor nucleic acid (e.g., an rAAV) to an individual. For example, in some cases fludarabine is administered in a range of from about 10 days to about 30 days (e.g., in a range of from 10 days to 25 days, 10 days to 21 days, 14 days to 30 days, 14 days to 25 days, or 14 days to 21 days) after administration of a donor nucleic acid (e.g., an rAAV) to an individual.

In some cases, a subject RNR inhibitor (e.g., hydroxyurea (HU), motexafin gadolinium, fludarabine, cladribine, gemcitabine, tezacitabine, triapine, gallium maltolate, and the like) is administered in a range of from about 10 days to about 25 days (e.g., in a range of from 10 days to 21 days, 14 days to 25 days, or 14 days to 21 days) after administration of a donor nucleic acid (e.g., an rAAV) to an individual. For example, in some cases fludarabine is administered in a range of from about 10 days to about 25 days (e.g., in a range of from 10 days to 21 days, 14 days to 25 days, or 14 days to 21 days) after administration of a donor nucleic acid (e.g., an rAAV) to an individual.

In some cases, a subject RNR inhibitor (e.g., hydroxyurea (HU), motexafin gadolinium, fludarabine, cladribine, gemcitabine, tezacitabine, triapine, gallium maltolate, and the like) is administered in a range of from about 2 weeks to about 3 weeks after administration of a donor nucleic acid (e.g., an rAAV) to an individual. For example, in some cases fludarabine is administered in a range of from about 2 weeks to about 3 weeks after administration of a donor nucleic acid (e.g., an rAAV) to an individual.

For some conditions, particularly central nervous system conditions, it may be necessary to formulate agents to cross the blood-brain bather (BBB). One strategy for drug delivery through the blood-brain barrier (BBB) entails disruption of the BBB, either by osmotic means such as mannitol or leukotrienes, or biochemically by the use of vasoactive substances such as bradykinin The potential for using BBB opening to target specific agents to brain tumors is also an option. A BBB disrupting agent can be co-administered with the therapeutic compositions of the disclosure when the compositions are administered by intravascular injection. Other strategies to go through the BBB may entail the use of endogenous transport systems, including Caveolin-1 mediated transcytosis, carrier-mediated transporters such as glucose and amino acid carriers, receptor-mediated transcytosis for insulin or transferrin, and active efflux transporters such as p-glycoprotein. Active transport moieties may also be conjugated to therapeutic compounds to facilitate transport across the endothelial wall of the blood vessel. Alternatively, drug delivery of therapeutics agents behind the BBB may be by local delivery, for example by intrathecal delivery, e.g. through an Ommaya reservoir (see e.g. U.S. Pat. Nos. 5,222,982 and 5,385,582, incorporated herein by reference); by bolus injection, e.g. by a syringe, e.g. intravitreally or intracranially; by continuous infusion, e.g. by cannulation, e.g. with convection (see e.g. US Application No. 20070254842, incorporated here by reference); or by implanting a device upon which the agent has been reversably affixed (see e.g. US Application Nos. 20080081064 and 20090196903, incorporated herein by reference).

Typically, an effective amount of an RNR inhibitor and an rAAV is provided. Both reagents can be delivered together as part of the same formulation or as part of separate formulations. When part of separate formulations, in some cases the formulations are administered at the same time, while in other cases the RNR inhibitor is administered prior to administration of the rAAV. Both reagents can be delivered using the same technique/route of delivery or can be delivered using different approaches (e.g., one might be delivered orally while the other is delivered via iv).. As discussed above with regard to ex vivo methods, an effective amount or effective dose of a targeting vector in vivo is the amount to induce an increase (e.g. in some cases a 2-fold increase or more) in the number of cells in which recombination between the targeting vector and the target locus can be observed relative to a negative control, e.g. a cell contacted with an empty vector or irrelevant polypeptide. The amount of recombination may be measured by any convenient method, e.g. as described above and known in the art. The calculation of the effective amount or effective dose of a targeting vector to be administered is within the skill of one of ordinary skill in the art, and will be routine to those persons skilled in the art. Needless to say, the final amount to be administered will be dependent upon the route of administration and upon the nature of the disorder or condition that is to be treated.

For inclusion in a medicament, an RNR inhibitor and an rAAV may be obtained from a suitable commercial source. As a general proposition, the total pharmaceutically effective amount of an RNR inhibitor and an rAAV per dose will be in a range that can be measured by a dose response curve.

In cases a therapy must be sterile (e.g., when treating a human patient). Sterility is readily accomplished by filtration through sterile filtration membranes (e.g., 0.2 μm membranes). Therapeutic compositions generally are placed into a container having a sterile access port, for example, an intravenous solution bag or vial having a stopper pierceable by a hypodermic injection needle. The RNR inhibitor and rAAV based therapies may be stored in unit or multi-dose containers, for example, sealed ampules or vials, as an aqueous solution or as a lyophilized formulation for reconstitution. As an example of a lyophilized formulation, 10-mL vials are filled with 5 ml of sterile-filtered 1% (w/v) aqueous solution of compound, and the resulting mixture is lyophilized. The infusion solution is prepared by reconstituting the lyophilized compound using bacteriostatic Water-for-Injection.

Pharmaceutical compositions can include, depending on the formulation desired, pharmaceutically-acceptable, non-toxic carriers of diluents, which are defined as vehicles commonly used to formulate pharmaceutical compositions for animal or human administration. The diluent is selected so as not to affect the biological activity of the combination. Examples of such diluents are distilled water, buffered water, physiological saline, PBS, Ringer's solution, dextrose solution, and Hank's solution. In addition, the pharmaceutical composition or formulation can include other carriers, adjuvants, or non-toxic, nontherapeutic, nonimmunogenic stabilizers, excipients and the like. The compositions can also include additional substances to approximate physiological conditions, such as pH adjusting and buffering agents, toxicity adjusting agents, wetting agents and detergents.

The composition can also include any of a variety of stabilizing agents, such as an antioxidant for example. When the pharmaceutical composition includes a polypeptide, the polypeptide can be complexed with various well-known compounds that enhance the in vivo stability of the polypeptide, or otherwise enhance its pharmacological properties (e.g., increase the half-life of the polypeptide, reduce its toxicity, enhance solubility or uptake). Examples of such modifications or complexing agents include sulfate, gluconate, citrate and phosphate. The nucleic acids or polypeptides of a composition can also be complexed with molecules that enhance their in vivo attributes. Such molecules include, for example, carbohydrates, polyamines, amino acids, other peptides, ions (e.g., sodium, potassium, calcium, magnesium, manganese), and lipids.

Further guidance regarding formulations that are suitable for various types of administration can be found in Remington's Pharmaceutical Sciences, Mace Publishing Company, Philadelphia, Pa., 17th ed. (1985). For a brief review of methods for drug delivery, see, Langer, Science 249:1527-1533 (1990).

The pharmaceutical compositions can be administered for prophylactic and/or therapeutic treatments. Toxicity and therapeutic efficacy of the active ingredient can be determined according to standard pharmaceutical procedures in cell cultures and/or experimental animals, including, for example, determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred.

In some embodiments, a pharmaceutical composition administered to a subject in an effective amount exhibits little to no liver toxicity (e.g., exhibits no substantial liver toxicity, does not exhibit substantial liver toxicity, is substantially non-toxic to the liver, etc.). Liver toxicity may be measured in a variety of ways, such as measuring levels of one, both, or a ratio of alanine aminotransferase (ALT) and aspartate aminotransferases (ASP). In some embodiments, administering an effective amount of the pharmaceutical composition induces an increase in liver toxicity (e.g., as measured by a selected convenient assay) of less than 50% (e.g., less than 40%, less than 30%, less than 20%, less than 15%, less than 10%, less than 5%, less than 4%, less than 3%, less than 2%, less than 1%, less than 0.5%, or 0%) as compared to such measure of liver toxicity prior to such administration (or as compared to an untreated control or as compared to an accepted normal range of values, i.e., reference values, for the measure). In some embodiments, administering an effective amount of the pharmaceutical composition induces no statistically significant increase in the measure of liver toxicity (e.g. at a p-value of less than 0.1, 0.05, 0.01, or lower) as compared to such measure prior to such administration (or as compared to an untreated control or as compared to an accepted normal range of values, i.e., reference values, for the measure). In some embodiments, administering an effective amount of the pharmaceutical composition reduces a measure of liver toxicity (e.g., as may result when the condition treated by the administration was causing liver toxicity) by 5% or more (e.g., 10% or more, 15% or more, 20% or more, 30% or more, 40% or more, 50% or more, etc.) as compared to such measure prior to such administration (or as compared to an untreated control or as compared to an accepted normal range of values, i.e., reference values, for the measure).

The data obtained from cell culture and/or animal studies can be used in formulating a range of dosages for humans. The dosage of the active ingredient typically lines within a range of circulating concentrations that include the ED50 with low toxicity. The dosage can vary within this range depending upon the dosage form employed and the route of administration utilized.

The components used to formulate the pharmaceutical compositions are preferably of high purity and are substantially free of potentially harmful contaminants (e.g., at least National Food (NF) grade, generally at least analytical grade, and more typically at least pharmaceutical grade). Moreover, compositions intended for in vivo use are usually sterile. To the extent that a given compound must be synthesized prior to use, the resulting product is typically substantially free of any potentially toxic agents, particularly any endotoxins, which may be present during the synthesis or purification process. Compositions for parental administration are also sterile, substantially isotonic and made under GMP conditions.

The effective amount of a therapeutic composition to be given to a particular patient will depend on a variety of factors, several of which will differ from patient to patient. A competent clinician will be able to determine an effective amount of a therapeutic agent to administer to a patient to halt or reverse the progression the disease condition as required. Utilizing LD50 animal data, and other information available for the agent, a clinician can determine the maximum safe dose for an individual, depending on the route of administration. For instance, an intravenously administered dose may be more than an intrathecally administered dose, given the greater body of fluid into which the therapeutic composition is being administered. Similarly, compositions which are rapidly cleared from the body may be administered at higher doses, or in repeated doses, in order to maintain a therapeutic concentration. Utilizing ordinary skill, the competent clinician will be able to optimize the dosage of a particular therapeutic in the course of routine clinical trials.

### Nucleotide Sequence Cassette

As noted above, the targeting vector will include a donor DNA having a nucleotide sequence cassette (a nucleotide sequence of interest) flanked by homology arms (sequences of homology to the target integration site). The nucleotide sequence cassette will include a transgene sequence. In some cases a subject sequence cassette will also include, 5′ or 3′ of the transgene sequence, a nucleic acid sequence that promotes the production of two independent gene products. The subject methods and compositions find particular use in inserting a transgene sequence into a targeted cell's genome at a target locus.

In some instances, the transgene sequence encodes an RNA that codes for a peptide or polypeptide. In other instances, the transgene sequence encodes for a non-coding RNA, i.e. an RNA that does not encode a peptide or protein, e.g. a nucleic acid sequence that encodes for a ribozyme, a small hairpin RNA (shRNA), a microRNA (miRNA), or a precursor thereof, a long-noncoding RNA, etc.

In some instances, one transgene sequence is inserted into the target locus. In other instances, more than one transgene sequences are inserted, e.g. 2, 3, 4, or 5 or more transgene sequences are inserted into the target locus. In some instances, the subject transgene sequence(s) becomes operably linked to the promoter of the endogenous gene at the target locus upon integration into the target integration site. In other instances, the subject transgene is operably linked to a promoter on the donor DNA, and remains operably linked to that promoter upon integration into the target integration site.

### Target Locus

The subject rAAVs are configured to guide the integration of the transgene sequence to a specific locus of interest, i.e., a “target locus”, in the cell genome. In other words, the integration is a targeted integration. The genomic targeted by the methods described herein can be any convenient locus. Examples of target loci of particular interest for integrating a transgene sequence (e.g., one encoding a protein, on encoding a non-coding RNA) include, but are not limited to: glyceraldehyde-3-phosphate dehydrogenase (GAPDH), an actin gene (e.g., alpha actin, beta actin, etc.), adenosine deaminase (ADA), albumin, ApoA2, α-globin, γ-globin, CD2, CD3, CDS, CD7, E1α, IL2RG, Ins1, Ins2, NCF1, p50, p65, PF4, PGC-γ, PTEN, TERT, UBC, VWF, and a collagen gene (e.g. collagen type 1, collagen type 2, collagen type 3, collagen type 4, collagen type 5, collagen type 6, collagen type 7, collagen type 8, collagen type 9, collagen type 10, collagen type 11, collagen type 12, collagen type 13, collagen type 14, collagen type 15, collagen type 16, collagen type 17, collagen type 18, collagen type 19, collagen type 20, collagen type 21, collagen type 22, collagen type 23, collagen type 24, collagen type 25, collagen type 26, collagen type 27, collagen type 28). Any convenient location within a target locus may be targeted, the donor DNA being configured to provide for targeted integration, e.g., in some cases without disrupting the aforementioned gene.

### Homology Arms and Target Loci

To promote targeted integration, the donor DNA comprises nucleic acid sequences (homology arms) that are permissive to homologous recombination at the site of integration, e.g. sequences that are permissive to homologous recombination with the albumin gene, a collagen gene, an actin gene, etc. This process requires nucleotide sequence homology, using the “donor” molecule, e.g. the targeting vector, to template repair of a “target” molecule, i.e., the nucleic acid into which the nucleic acid of sequence is integrated, e.g. a target locus in the cellular genome, and leads to the transfer of genetic information from the donor to the target. As such, in donor DNAs of the subject compositions, the nucleotide sequence cassette (e.g., a transgene sequence only, a transgene sequence adjacent to a 2A peptide sequence, a transgene sequence plus a promoter to which it is operably linked, etc.) to be integrated into the cellular genome may be flanked by sequences that contain sufficient homology to a genomic sequence at the cleavage site, e.g. 70%, 80%, 85%, 90%, 95%, or 100% homology with the nucleotide sequences flanking the cleavage site, e.g. within about 50 bases or less of the cleavage site, e.g. within about 30 bases, within about 15 bases, within about 10 bases, within about 5 bases, or immediately flanking the target integration site, to support homologous recombination between it and the genomic sequence to which it bears homology. Approximately 25, 50, 100, 250, or 500 nucleotides or more of sequence homology between a donor and a genomic sequence will support homologous recombination therebetween.

In some embodiments, the presence of the flanking sequences that are permissive to homologous recombination provide for an increased rate of target site integration, as compared to a vector lacking the flanking sequences or having flanking sequences that are not homologous to the target locus (e.g., flanking sequences that are homologous to a different genomic locus, flanking sequences with no homology to any location in the target genome, etc.). In some embodiments, 0.01% or more (e.g., 0.05% or more, 0.1% or more, 0.2% or more, 0.3% or more, 0.4% or more, 0.5% or more, 0.6% or more, 0.7% or more, 0.8% or more, 0.9% or more, 1% or more, 1.5% or more, 2% or more, 5% or more, 10% or more) of target loci among cells in a tissue or among cells receiving the targeting vector contain an integrated transgene following administration. Rate of integration into a target locus may be measured by any suitable assay (e.g., a linear amplification assay).

In some embodiments, transgene expression results substantially from integration at the target locus. For example, in some cases 75% or more (e.g., 80% or more, 85% or more, 90% or more, 95% or more, 99% or more, 99.5% or more) of the total transgene expression is from the transgene that has integrated at the target locus. In other words, in some cases, the relative fraction of transgene expression from sources other than integration at the target locus (e.g. episomal expression, or integration at a non-target locus) as compared to expression from integration at the target locus is 25% or less (e.g., 20% or less, 15% or less, 10% or less, 5% or less, 1% or less, 0.5% or less, etc.). The percent of expression from target-locus-based integration can be measured by any suitable assay, e.g., an assay disclosed herein.

The flanking recombination sequences can be of any length, e.g. 10 nucleotides or more, 50 nucleotides or more, 100 nucleotides or more, 250 nucleotides or more, 500 nucleotides or more, 1000 nucleotides (1 kb) or more, 5000 nucleotides (5 kb) or more, 10000 nucleotides (10 kb) or more etc. Generally, the homologous region(s) of a donor sequence will have at least 50% sequence identity to a genomic sequence with which recombination is desired. In certain embodiments, 60%, 70%, 80%, 90%, 95%, 98%, 99%, or 99.9% sequence identity is present. Any value between 1% and 100% sequence identity can be present, depending upon the length of the targeting vector.

In some instances, the flanking sequences (homology arms) may be substantially equal in length to one another, e.g. one may be 30% shorter or less than the other flanking sequence, 20% shorter or less than the other flanking sequence, 10% shorter or less than the other flanking sequence, 5% shorter or less than the other flanking sequence, 2% shorter or less than the other flanking sequence, or only a few nucleotides less than the other. In other instances, the flanking sequences may be substantially different in length from one another, e.g. one may be 40% shorter or more, 50% shorter or more, sometimes 60% shorter or more, 70% shorter or more, 80% shorter or more, 90% shorter or more, or 95% shorter or more than the other flanking sequence.

Often, at least one flanking recombination sequence will comprise coding sequence for the gene at the target locus. For example, if the target integration site comprises the 3′ end of the endogenous gene, the recombination sequence on the targeting vector that is 5′ of the transgene will be substantially homologous to DNA sequence upstream of, e.g. adjacent to, the stop codon of the endogenous gene, while the recombination sequence on the targeting vector that is 3′ of the transgene will be substantially homologous to the DNA sequence downstream of, e.g. adjacent to, the stop codon of the endogenous gene. As another example, if the target integration site comprises the 5′ end of the endogenous gene, the recombination sequence on the targeting vector that is 5′ of the transgene will be substantially homologous to the DNA sequence upstream of, e.g. adjacent to, the start codon of the endogenous gene, while the recombination sequence on the targeting vector that is 3′ of the transgene will be substantially homologous to the DNA sequence downstream of, e.g. adjacent to, the start codon of the endogenous gene. Integrating coding sequence for the gene at the target locus into the target locus finds many uses. For example, integrating coding sequence for the gene at the target locus that is downstream, or 3′, of the insertion site will ensure that the expression of the gene is not substantially disrupted by the integration of the gene of interest. As another example, it may be desirable to integrate coding sequence for the gene at the target locus so as to express a gene sequence that is a variant from that at the cell's target locus, e.g. if the gene at the cell's target locus is mutant, e.g. to complement a mutant target locus with wild-type gene sequence to treat a genetic disorder.

The methods and compositions disclosed herein find use in any in vitro or in vivo application in which it is desirable to express a transgene from a particular locus in a cell, for example when it is desirable to express one or more transgenes in a cell in the same spatially and temporally restricted pattern as that of an endogenous gene at a target locus, while maintaining the expression of that endogenous gene at that target locus (in some cases avoiding the risk of using an exogenous nuclease).

The subject methods and compositions for integrating a sequence cassette into cellular DNA at a target locus finds use in many fields, including, for example, gene therapy, agriculture, biotechnology, and research. For example, such modifications are therapeutically useful, e.g. to treat a genetic disorder by complementing a genetic mutation in a subject with a wild-type copy of the gene; to promote naturally occurring processes, by promoting/augmenting cellular activities (e.g. promoting wound healing for the treatment of chronic wounds or prevention of acute wound or flap failure, by augmenting cellular activities associated with wound healing); to modulate cellular response (e.g. to treat diabetes mellitus, by providing insulin); to express antiviral. antipathogenic, or anticancer therapeutics in subjects, e.g. in specific cell populations or under specific conditions, etc. Other uses for such genetic modifications include in the induction of induced pluripotent stem cells (iPSCs), e.g. to produce iPSCs from an individual for diagnostic, therapeutic, or research purposes; in the production of genetically modified organisms, for example in manufacturing for the large scale production of proteins by cells for therapeutic, diagnostic, or research purposes; in agriculture, e.g. for the production of improved crops; or in research, e.g. for the study of animal models of disease.

For example, the subject methods and compositions may be used to treat a disorder, a disease, or medical condition in a subject. Towards this end, the one or more transgene sequences of the subject compositions may include a gene that encodes a therapeutic agent. By a “therapeutic agent” it is meant an agent, e.g. ribozyme, siRNA, shRNA, miRNA, peptide, polypeptide, etc. that has a therapeutic effect upon a cell or an individual, for example, that promotes a biological process to treat a medical condition, e.g. a disease or disorder.

Examples of therapeutic agents that may be integrated into a cellular genome using the subject methods and compositions include (i.e., the integrated transgene encodes) agents such as ribozymes, siRNAs, shRNAs, miRNAs, peptides (e.g., a nucleic acid encoding a peptide), or polypeptides (e.g., a nucleic acid encoding a polypeptide) which alter cellular activity. In some instances, the transgene encodes a peptide or polypeptide. Example of peptide or polypeptides envisioned as having a therapeutic activity for the multicellular organism in which they are expressed (e.g., via a nucleic acid encoding the peptide or polypeptide) include, but are not limited to: factor VIII, factor IX, β-globin, a CRISPR/Cas effector protein (e.g., Cas9, Cpf1, and the like), a low-density lipoprotein receptor, adenosine deaminase, purine nucleoside phosphorylase, sphingomyelinase, glucocerebrosidase, cystic fibrosis transmembrane conductance regulator, α1-antitrypsin, CD-18, PDGF, VEGF, EGF, TGFα, TGBβ, FGF, TNF, IL-1, IL-2, IL-6, IL-8, endothelium derived growth factor (EDGF), ornithine transcarbamylase, argininosuccinate synthetase, phenylalanine hydroxylase, branched-chain α-ketoacid dehydrogenase, fumarylacetoacetate hydrolase, glucose 6-phosphatase, α-L-fucosidase, (3-glucuronidase, α-L-iduronidase, galactose 1-phosphate uridyltransferase; a neuroprotective factor, e.g. a neurotrophin (e.g. NGF, BDNF, NT-3, NT-4, CNTF), Kifap3, Bcl-xl, collapsin response mediator protein 1, Chkβ, calmodulin 2, calcyon, NPT1, Eef1a1, Dhps, Cd151, Morf412, CTGF, LDH-A, Atli, NPT2, Ehd3, Cox5b, Tuba1a, γ-actin, Rpsa, NPG3, NPG4, NPG5, NPG6, NPG7, NPG8, NPG9, NPG10, dopamine, interleukins, cytokines, small peptides, the genes/proteins listed in Table 1 (see below: BCKDH complex (E1α, Elb and E2 subunits); Methylmalonyl-CoA Mutase; Propionyl-CoA Carboxylase (Alpha and Beta subunits); Isovaleryl CoA dehydrogenase; HADHA; HADHB; LCHAD; ACADM; ACADVL; G6PC (GSD1a); G6PT1(GSD1b); SLC17A3; SLC37A4 (GSD1c); Acid alpha-glucosidase; OCTN2; CPT1; CACT; CPT2; CPS1; ARG1; ASL; OTC; UGT1A1; FAH; COL7A1; COL17A1; MMP1; KRT5; LAMA3; LAMB3; LAMC2; ITGB4; and/or ATP7B), and the like. The above list of proteins refers to mammalian proteins, and in many embodiments human proteins, where the nucleotide and amino acid sequences of the above proteins are generally known to those of skill in the art.

TABLE 1
List of genes/proteins that are defective in various diseases

| Family of diseases | Diseases | Gene/protein |
|---|---|---|
| Branched-chain | Maple Syrup Urine | BCKDH complex |
| organic acidurias | Disease (MSUD) | (E1a, E1b and E2 |
| | | subunits) |
| | Methylmalonic | Methylmalonyl-CoA |
| | Acidemia (MMA) | Mutase |
| | Propionic Acidemia | Propionyl-CoA |
| | (PA) | Carboxylase (Alpha |
| | | and Beta subunits) |
| | IsoValeric Acidemia | Isovaleryl CoA |
| | (IVA) | dehydrogenase |
| Long chained fatty | trifunctional | HADHA and HADHB |
| acid oxidation | protein deficiency | |
| disorders | LCHADD | LCHAD |
| | MCHADD | ACADM |
| | VLCHADD | ACADVL |
| Glycogen | GSD1 | G6PC (GSD1a), |
| storage disease | | G6PT1(GSD1b), |
| | | SLC17A3 or SLC37A4 |
| | | (GSD1c) |
| | GSD2 | Acid alpha-glucosidase |
| Carnitine cycle disorders | | OCTN2 |
| | | CPT1 |
| | | CACT |
| | | CPT2 |
| Urea cycle disorders | | CPS1 |
| | | ARG1 |
| | | ASL |
| | | OTC |
| | Crigler-Najjar syndrome | UGT1A1 |
| | Heraditary Tyrosinemia | FAH |
| | Epidermolysis Bullosa | COL7A1 or COL17A1 |
| | | or MMP1 or KRT5 |
| | | or LAMA3 or LAMB3 |
| | | or LAMC2 or ITGB4 |
| | Wilson Disease | ATP7B |

In other instances, the transgene encodes for an RNA that does not encode a protein, e.g.

the nucleic acid encodes for a ribozyme, a small hairpin RNA (shRNA), a microRNA (miRNA) or a precursor thereof, a guide RNA for a CRISPR/Cas effector protein, and the like. As used herein, the term “microRNA” refers to any type of interfering RNAs, including but not limited to, endogenous microRNAs and artificial microRNAs (e.g., synthetic miRNAs). Endogenous microRNAs are small RNAs naturally encoded in the genome which are capable of modulating the productive utilization of mRNA. An artificial microRNA can be any type of RNA sequence, other than endogenous microRNA, which is capable of modulating the activity of an mRNA. A microRNA sequence can be an RNA molecule composed of any one or more of these sequences. MicroRNA (or “miRNA”) sequences have been described in publications such as Lim, et al., 2003, Genes & Development, 17, 991-1008, Lim et al., 2003, Science, 299, 1540, Lee and Ambrose, 2001, Science, 294, 862, Lau et al., 2001, Science 294, 858-861, Lagos-Quintana et al., 2002, Current Biology, 12, 735-739, Lagos-Quintana et al., 2001, Science, 294, 853-857, and Lagos-Quintana et al., 2003, RNA, 9, 175-179. Examples of microRNAs include any RNA that is a fragment of a larger RNA or is a miRNA, siRNA, stRNA, sncRNA, tncRNA, snoRNA, smRNA, shRNA, snRNA, or other small non-coding RNA. See, e.g., US Patent Applications 20050272923, 20050266552, 20050142581, and 20050075492. A “microRNA precursor” (or “pre-miRNA”) refers to a nucleic acid having a stem-loop structure with a microRNA sequence incorporated therein. A “mature microRNA” (or “mature miRNA”) includes a microRNA that has been cleaved from a microRNA precursor (a “pre-miRNA”), or that has been synthesized (e.g., synthesized in a laboratory by cell-free synthesis), and has a length of from about 19 nucleotides to about 27 nucleotides, e.g., a mature microRNA can have a length of 19 nt, 20 nt, 21 nt, 22 nt, 23 nt, 24 nt, 25 nt, 26 nt, or 27 nt. A mature microRNA can bind to a target mRNA and inhibit translation of the target mRNA.

Other examples of therapeutic agents that may be integrated into a target locus include (i.e., the integrated transgene encodes) agents that promote immunoprophylaxis (also referred to as vectored immunoprophylaxis, or VIP). Examples of agents that promote immunoprophylaxis include, but are not limited to: antibodies or chimeric polypeptides comprising an immunoglobulin domain and an immune effector domain As non-limiting examples, agents that promote immunoprophylaxis can include neutralizing antibodies, or chimeric polypeptides, specific for a pathogen selected from: human immunodeficiency virus (HIV), influenza virus, Respiratory Syncytial Virus (RSV), Hepatitis C virus (HCV), a plasmodium (e.g., Plasmodium falciparum, plasmodium malariae, and the like), fungal or bacterial pathogens, and the like. For example, agents that promote immunoprophylaxis can include neutralizing antibodies, or chimeric polypeptides, that target epitopes conserved among strains of human immunodeficiency virus (HIV), influenza virus, Respiratory Syncytial Virus (RSV), Hepatitis C virus (HCV), a plasmodium (e.g., Plasmodium falciparum, plasmodium malariae, and the like), fungal or bacterial pathogens, and the like.

In some instances, the therapeutic agent alters the activity of the cell in which the agent is expressed. In other words, the agent has a cell-intrinsic effect. For example, the agent may be an intracellular protein, transmembrane protein or secreted protein that, when expressed in a cell, will substitute for, or “complement”, a mutant protein in the cell. In other instances, the therapeutic agent alters the activity of cells other than cells in which the agent is expressed. In other words, the agent has a cell-extrinsic effect. For example, the integrated gene of interest may encode a cytokine, chemokine, growth factor, hormone, antibody, or cell surface receptor that modulates the activity of other cells.

The subject methods and compositions may be applied to any disease, disorder, or natural cellular process that would benefit from modulating cell activity by integrating a transgene of interest. For example, the subject methods and compositions find use in treating genetic disorders. Any genetic disorder that results from a defined genetic defect (e.g., a disorder with a single gene defect, a disorder with 2 defective genes, 3 defective genes, 4 defective genes, 5 defective genes, 2 or more defective genes, 3 or more defective genes, 4 or more defective genes, 5 or more defective genes, etc.) may be treated by the subject compositions and methods. The defect may result from one or more mutations in a single gene (e.g. 1, 2, 3, 4, 5, or more mutations), or may result from one or more mutations in 2 or more genes (e.g., 3 or more genes, 4 or more genes, 5 or more genes, 2 genes, 3 genes, 4 genes, 5 genes, etc.). Non-limiting examples of diseases resulting from genetic defects include: hemophilia (e.g., hemophilia A, hemophilia B), branched-chain organic acidurias (e.g., Maple syrup urine disease (MSUD), isovaleric acidaemia (IVA), propionic aciduria (PA) and methylmalonic aciduria (MMA), 3-methylcrotonyl glycinuria, 3-methylglutaconic Aciduria Type I, Short/branched-chain Acyl-CoA Dehydrogenase Deficiency, 2-methyl-3-hydroxybutyryl-CoA Dehydrogenase Deficiency, Isobutyryl-CoA Dehydrogenase Deficiency, 3-Hydroxyisobutyric Aciduria, Malonic Aciduria, etc.), long chained fatty acid oxidation disorders, glycogen storage diseases (e.g., glycogen storage disease type I (GSD1), glycogen storage disease type II, glycogen storage disease type III, glycogen storage disease type IV, glycogen storage disease type V, glycogen storage disease type VI, glycogen storage disease type VII, glycogen storage disease type VIII, glycogen storage disease type IX, glycogen storage disease type X, glycogen storage disease type XI, glycogen storage disease type XII, glycogen storage disease type 0, etc.), carnitine cycle disorders, urea cycle disorders, Crigler-Najjar syndrome, Heraditary Tyrosinemia, Epidermolysis Bullosa, Wilson Disease, adenosine deaminase deficiency, sickle cell disease, X-Linked Severe Combined Immunodeficiency (SCID-X1), thalassemia, cystic fibrosis, alpha-1 anti-trypsin deficiency, diamond-blackfan anemia, Gaucher's disease, growth hormone deficiency, and the like.

As another example, the subject methods and compositions find use in treating nervous system conditions and to protect the CNS against nervous system conditions, e.g. neurodegenerative diseases, including, for example, e.g. Parkinson's Disease, Alzheimer's Disease, Huntington's Disease, Amyotrophic Lateral Sclerosis (ALS), Spielmeyer-Vogt-Sjogren-Batten disease (Batten Disease), Frontotemporal Dementia with Parkinsonism, Progressive Supranuclear Palsy, Pick Disease, prion diseases (e.g. Creutzfeldt-Jakob disease), Amyloidosis, glaucoma, diabetic retinopathy, age related macular degeneration (AMD), and the like); neuropsychiatric disorders (e.g. anxiety disorders (e.g. obsessive compulsive disorder), mood disorders (e.g. depression), childhood disorders (e.g. attention deficit disorder, autistic disorders), cognitive disorders (e.g. delirium, dementia), schizophrenia, substance related disorders (e.g. addiction), eating disorders, and the like); channelopathies (e.g. epilepsy, migraine, and the like); lysosomal storage disorders (e.g. Tay-Sachs disease, Gaucher disease, Fabry disease, Pompe disease, Niemann-Pick disease, Mucopolysaccharidosis (MPS) & related diseases, and the like); autoimmune diseases of the CNS (e.g. Multiple Sclerosis, encephalomyelitis, paraneoplastic syndromes (e.g. cerebellar degeneration), autoimmune inner ear disease, opsoclonus myoclonus syndrome, and the like); cerebral infarction, stroke, traumatic brain injury, and spinal cord injury.

As another for example, the subject methods and compositions may be used in the treatment of medical conditions and diseases in which it is desirable to ectopically express a therapeutic agent to promote tissue repair, tissue regeneration, or protect against further tissue insult, e.g. to promote wound healing; promote the survival of the cell and/or neighboring cells, e.g. in degenerative disease, e.g. neurodegenerative disease, kidney disease, liver disease, etc.; prevent or treat infection, etc.

Other examples of how the subject methods may be used to treat medical conditions are disclosed elsewhere herein, or would be readily apparent to the ordinarily skilled artisan.

The subject methods and compositions also find us in imaging cells of interest, e.g. cells comprising an integrated gene of interest. As such, the transgene (or one of the transgenes) to be integrated may encode for an imaging marker. By an “imaging marker” it is meant a non-cytotoxic agent that can be used to locate and, optionally, visualize cells, e.g. cells that have been targeted by compositions of the subject application. An imaging moiety may require the addition of a substrate for detection, e.g. horseradish peroxidase (HRP), β-galactosidase, luciferase, and the like. Alternatively, an imaging moiety may provide a detectable signal that does not require the addition of a substrate for detection, e.g. a fluorophore or chromophore dye, e.g. Alexa Fluor 488® or Alexa Fluor 647®, or a protein that comprises a fluorophore or chromophore, e.g. a fluorescent protein. As used herein, a fluorescent protein (FP) refers to a protein that possesses the ability to fluoresce (i.e., to absorb energy at one wavelength and emit it at another wavelength). For example, a green fluorescent protein (GFP) refers to a polypeptide that has a peak in the emission spectrum at 510 nm or about 510 nm. A variety of FPs that emit at various wavelengths are known in the art. FPs of interest include, but are not limited to, a green fluorescent protein (GFP), yellow fluorescent protein (YFP), orange fluorescent protein (OFP), cyan fluorescent protein (CFP), blue fluorescent protein (BFP), red fluorescent protein (RFP), far-red fluorescent protein, or near-infrared fluorescent protein and variants thereof.

As another example, the subject methods and compositions find use in isolating cells of interest, e.g. cells comprising an integrated transgene. Towards this end, the transgene (or one of the transgenes) to be integrated may encode for a selectable marker. By a “selectable marker” it is meant an agent that can be used to select cells, e.g. cells that have been targeted by compositions of the subject application. In some instances, the selection may be positive selection; that is, the cells are isolated from a population, e.g. to create an enriched population of cells comprising the genetic modification. In other instances, the selection may be negative selection; that is, the population is isolated away from the cells, e.g. to create an enriched population of cells that do not comprise the genetic modification. Any convenient selectable marker may be employed, for example, a drug selectable marker, e.g. a marker that prevents cell death in the presence of drug, a marker that promotes cell death in the presence of drug, an imaging marker, etc.; an imaging marker that may be selected for using imaging technology, e.g. fluorescence activated cell sorting; a polypeptide or peptide that may be selected for using affinity separation techniques, e.g. fluorescence activated cell sorting, magnetic separation, affinity chromatography, “panning” with an affinity reagent attached to a solid matrix, etc.; and the like.

In some instances, the transgene may be conjugated to a coding domain that modulates the stability of the encoded protein, e.g. in the absence/presence of an agent, e.g. a cofactor or drug. Non-limiting examples of destabilizing domains that may be used include a mutant FRB domain that is unstable in the absence of rapamycin-derivative C20-MaRap (Stankunas K, et al. (2003) Conditional protein alleles using knockin mice and a chemical inducer of dimerization. Mol Cell. 12(6):1615-24); an FKBP12 mutant polypeptide that is metabolically unstable in the absence of its ligand Shield-1 (Banaszynski L A, et al. (2006) A rapid, reversible, and tunable method to regulate protein function in living cells using synthetic small molecules. Ce11.126(5):995-1004); a mutant E. coli dihydrofolate reductase (DHFR) polypeptide that is metabolically unstable in the absence of trimethoprim (TMP) (Mari Iwamoto, et al. (2010) A general chemical method to regulate protein stability in the mammalian central nervous system. Chem Biol. 2010 Sep. 24; 17(9): 981-988); and the like.

As discussed above, any nucleic acid sequence that the ordinarily skilled artisan would like expressed in a cell may be integrated into a target locus, for example, any nucleic acid sequence encoding a non-coding RNA such as, e.g., a ribozyme, siRNA, shRNA, miRNA, or long-noncoding RNA; or any nucleic acid sequence encoding an RNA coding for a peptide or polypeptide, may be integrated. In some instances, more than one sequence to be expressed may be integrated, for example, two or more polynucleotides of interest may be integrated, three or more polynucleotides may be integrated, four or more polynucleotides may be integrated, e.g. five or more polynucleotides may be integrated. Thus, for example, a therapeutic gene and an imaging marker may be integrated; a therapeutic gene and a non-coding RNA may be integrated; a therapeutic gene and a selectable marker may be integrated, an imaging marker and a selectable marker may be integrated, a therapeutic gene, an imaging marker and a selectable marker may be integrated, and so forth.

Sequences promoting production of two independent gene products

In some embodiments, it is desirable to edit the genome of the cell without substantially disrupting the expression of the gene at the edited locus. Towards this end, the nucleotide sequence cassette that is integrated into the genome may include one or more additional nucleic acid sequences that provide for the expression of the transgene without substantially disrupting the expression of the gene at the target locus. For example, the targeting vector may comprise a nucleic acid sequence that promotes the production of two independent gene products—the endogenous gene at the target locus, and the integrated transgene seqeunce—upon integration of the transgene into the target integration site. Examples of such nucleic acid sequences include a sequence that encodes a 2A peptide; an IRES; an intein; a recognition sequence for a site specific protease (e.g. Furin), a sequence that encodes a cleavable linker that is cleaved as part of the coagulation cascade; a sequence that encodes a factor XI cleavage site; and intronic splice donor/splice acceptor sequences.

By a “2A peptide” it is meant a small (18-22 amino acids) peptide sequence that allows for efficient, concordant expression of discrete protein products within a single coding sequence, regardless of the order of placement of the genes within the coding sequence, through ribosomal skipping. 2A peptides are readily identifiable by their consensus motif (DVEXNPGP) and their ability to promote protein cleavage. Any convenient 2A peptide may be used in the targeting vector, e.g. the 2A peptide from a virus such as foot-and-mouth disease virus (F2A), equine Rhinitis A virus, porcine teschovirus-1 (P2A) or Thosea asigna virus (T2A), or any of the 2A peptides described in Szymczak-Workman, A. et al. “Design and Construction of 2A Peptide-Linked Multicistronic Vectors”. Adapted from: Gene Transfer: Delivery and Expression of DNA and RNA (ed. Friedmann and Rossi). CSHL Press, Cold Spring Harbor, N.Y., USA, 2007, the disclosure of which is incorporated herein by reference.

A transgene and 2A peptide coding sequence can be positioned on the targeting vector so as to provide for uninterrupted expression, i.e. transcription, translation, and activity, of the endogenous gene at the target locus upon insertion of the transgene sequence. For example, it may be desirable to insert the transgene sequence into an integration site near the 5′ end of the endogenous gene at the target locus, e.g., just downstream of the start codon of the endogenous gene at the target locus. In such instances, the 2A peptide coding sequence would be positioned within the targeting vector such that it is immediately 3′ to the transgene sequence, and flanking recombination sequences selected that will guide homologous recombination and integration of the transgene-2A peptide coding sequence cassette to the integration site just downstream of the start codon of the endogenous gene at the target locus. As another example, it may be desirable to insert the transgene sequence into an integration site within the 3′ end of the endogenous gene at the target locus, i.e. just upstream of the stop codon of the endogenous gene at the target locus. In such instances, the 2A peptide coding sequence would be positioned within the targeting vector such that it is immediately 5′ to the transgene sequence, and flanking recombination sequences selected that will guide homologous recombination and integration of the 2A-transgene cassette to the integration site just upstream of the stop codon of the endogenous gene at the target locus.

By an “internal ribosome entry site,” or “IRES” it is meant a nucleotide sequence that allows for the initiation of protein translation in the middle of a messenger RNA (mRNA) sequence. For example, when an IRES segment is located between two open reading frames in a bicistronic eukaryotic mRNA molecule, it can drive translation of the downstream protein-coding region independently of the 5′-cap structure bound to the 5′ end of the mRNA molecule, i.e. in front of the upstream protein coding region. In such a setup both proteins are produced in the cell. The protein located in the first cistron is synthesized by the cap-dependent initiation approach, while translation initiation of the second protein is directed by the IRES segment located in the intercistronic spacer region between the two protein coding regions. IRESs have been isolated from viral genomes and cellular genomes. Artificially engineered IRESs are also known in the art. Any convenient IRES may be employed in the donor polynucleotide.

Typically, as with the 2A peptide, the transgene and IRES will be positioned on the targeting vector so as to provide for uninterrupted expression of the gene at the target locus upon insertion of the transgene. For example, it may be desirable to insert the transgene into an integration site within the 5′ untranslated region (UTR) of the gene at the target locus. In such instances, the IRES would be positioned within the targeting vector such that it is immediately 3′ to the transgene sequence, and flanking recombination sequences selected that will guide homologous recombination and integration of the transgene-IRES cassette to the integration site within the 5′ UTR. As another example, it may be desirable to insert the transgene into an integration site within the 3′ UTR of the gene at the target locus, i.e. downstream of the stop codon, but upstream of the polyadenylation sequence. In such instances, the IRES would be positioned within the targeting vector such that it is immediately 5′ to the transgene sequence, and flanking recombination sequences selected that will guide homologous recombination and integration of the IRES-transgene cassette to the integration site within the 3′ UTR of the gene at the target locus.

By an “intein” it is meant a segment of a polypeptide that is able to excise itself and rejoin the remaining portions of the translated polypeptide sequence (the “exteins”) with a peptide bond. In other words, the targeting vector comprises nucleic acid sequences that, when translated, promote excision of the protein encoded by the transgene from the polypeptide that is translated from the modified target locus. Inteins may be naturally occurring, i.e. inteins that spontaneously catalyze a protein splicing reaction to excise their own sequences and join the flanking extein sequences, or artificial, i.e. inteins that have been engineered to undergo controllable splicing. Inteins typically comprise an N-terminal splicing region comprising a Cys (C), Ser (S), Ala (A), Gln (Q) or Pro (P) at the most N-terminal position and a downstream TXXH sequence; and a C-terminal splicing region comprising an Asn (N), Gln (Q) or Asp (D) at the most C-terminal position and a His (H) at the penultimate C-terminal position. In addition, a Cys (C), Ser (S), or Thr (T) is located in the +1 position of the extein from which the intein is spliced (−1 and +1 of the extein being defined as the positions immediately N-terminal and C-terminal, respectively, to the intein insertion site). Mechanism by which inteins promote protein splicing and the requirements for intein splicing may be found in Liu, X-Q, “Protein Splicing Intein: Genetic Mobility, Origin, and Evolution” Annual Review of Genetics 2000, 34: 61-76 and in publicly available databases such as, for example, the InBase database on the New England Biolabs website, found on the world wide web at “tools(dot)neb(dot)com/inbase/mech(dot)php”, the disclosures of which are incorporated herein by reference. Any sequences, e.g. N-terminal splicing regions and C-terminal splicing regions, known to confer intein-associated excision, be it spontaneous or controlled excision, on a donor polynucleotide, find use in the subject compositions. Genes of interest that are configured as inteins may be inserted at an integration site in any exon of a target locus, i.e. between the start codon and the stop codon of the gene at the target locus.

By a recognition sequence for a site specific protease, it is generally meant a nucleic acid sequence that encodes an amino acid sequence that is recognized by an enzyme that performs proteolysis. In some cases, such an amino acid sequence is referred to as a “cleavable linker.” For example, in some cases the cleavable linker is cleaved as part of the coagulation cascade (e.g., in some cases, the recognition sequence for a site specific protease is a factor XI cleavage site). Non-limiting examples of proteases that are highly specific and the sequences that they cleave include thrombin (cleaves after the arginine residue at its cleavage site Leu-Val-Pro-Arg-Gly-Ser), TEV protease (cleaves after the glutamine residue at its cleavage site Glu-X-X-Tyr-X-Gln-Ser), Furin (cleaves protein after the last arginine of the sequence Arg-X-(Lys/Arg)-Arg), Enterokinase (cleaves after the lysine residue at its cleavage site Asp-Asp-Asp-Asp-Lys); Factor Xa (cleaves after the arginine residue at its cleavage site Ile-(Glu or Asp)-Gly-Arg); Genenase I (cleaves at the site Pro-Gly-Ala-Ala-His-Tyr); HRV 3C protease (cleaves after the glutamine residue at its cleavage site Leu-Glu-Val-Leu-Phe-Gln-Gly-Pro). In some embodiments, the cleavable linker is cleaved by an intracellular protease. In some embodiments, the cleavable linker is cleaved by an extracellular protease.

By an “intron” it is meant any nucleotide sequence within a gene that is removed by RNA splicing to generate the final mature RNA product of a gene. In other words, the targeting vector comprises nucleic acid sequences that, when transcribed, promote excision of the pre-RNA encoded by the gene of interest from the pre-RNA that is transcribed from the modified target locus, allowing the transgene to be translated separately (or not, if the transgene encodes an siRNA, miRNA, etc.) from the mRNA of the target locus. Introns typically comprise a 5′ splice site (splice donor), a 3′ splice site (spice acceptor) and a branch site. The splice donor includes an almost invariant sequence GU at the 5′ end of the intron. The splice acceptor terminates the intron with an almost invariant AG sequence. Upstream (5′-ward) from the splice acceptor is a region high in pyrimidines (C and U) or a polypyrimidine tract. Upstream from the polypyrimidine tract is the branch point, which includes an adenine nucleotide. In addition to comprising these elements, the targeting vector may comprise one or more additional sequences that promote the translation of the mRNA transcribed from the gene of interest, e.g. a Kozak consensus sequence, a ribosomal binding site, an internal ribosome entry site, etc. Genes of interest that are configured as introns may be inserted at an integration site within the transcribed sequence of a target locus anywhere 5′ of the nucleic acid sequence that encodes the polyadenylation sequence, e.g. the 3′ untranslated region, the coding sequence, or the 5′ untranslated region of the gene at the target locus.

As discussed above, in some instances, it may be desirable to insert two or more transgene sequences of interest, e.g. 2 or more, 3 or more, 4 or more, or 5 or more transgene sequences of interest into a target locus. In such instances, multiple 2A peptides or IRESs may be used to create a bicistronic or multicistronic targeting vector. For example, a transgene and a selectable marker may be integrated into the 3′ region of the gene at the target locus, with 2A peptides being used to promote their cleavage from the polypeptide encoded by the target locus and from one another. Alternatively, coding sequences of interest may be provided on the targeting vector under the control of a promoter distinct from that of the gene at the target locus.

Typically, the gene of interest, the 2A peptide, and the recombination sequences will be positioned on the targeting vector so as to provide for uninterrupted expression of the gene at the target locus upon insertion of the gene of interest. For example, as discussed above, it may be desirable to insert the transgene into an integration site that is 3′, or “downstream” of the initiation codon of the gene at the target locus, for example, within the first 50 nucleotides 3′ of the initiation codon (i.e. the start ATG) for the gene at the target locus, e.g. within the first 25 nucleotides 3′ of initiation codon, within the first 10 nucleotides 3′ of the initiation codon, within the first 5 nucleotides 3′ of the initiation codon, or in some instances, immediately 3′ of the initiation codon, i.e. adjacent to the initiation codon. In such instances, the 2A peptide would be positioned within the targeting vector such that it is immediately 3′ to the gene of interest, and flanking recombination sequences selected that will guide homologous recombination and integration of the gene of interest to the integration site that is 3′ of the initiation codon at the target locus. As another example, it may be desirable to insert the gene of interest into an integration site that is 5′, or “upstream” of the termination codon of the gene at the target locus, for example, within the first 50 nucleotides 5′ of the termination codon (i.e. the stop codon, e.g. TAA, TAG, or TGA), e.g. within the first 25 nucleotides 5′ of termination codon, within the first 10 nucleotides 5′ of the termination codon, within the first 5 nucleotides of the termination codon, or in some embodiments, immediately 5′ of the termination codon, i.e. adjacent to the termination codon. In such instances, the 2A peptide would be positioned within the targeting vector such that it is immediately 5′ to the gene of interest, and flanking recombination sequences selected that will guide homologous recombination and integration of the gene of interest to the integration site that is 5′ of the termination codon at the target locus.

The targeting vector may also comprise sequences, e.g. restriction sites, nucleotide polymorphisms, selectable markers, etc., which may be used to assess for successful insertion of the gene of interest at the integration site. Typically, the targeting vector will also comprise a vector backbone containing sequences, e.g. viral sequences, e.g. replication origins, cap gene, rep gene, ITRs, etc., that are not homologous to the target region of interest and that are not intended for insertion into the target region of interest.

Nucleases

In some cases, a subject method includes the use of an exogenously provided site-specific nuclease. In some cases, a subject method does not include the use of an exogenously provided site-specific nuclease (a ‘targeted nuclease’). By a “nuclease” it is meant an enzyme that is capable of cleaving the phosphodiester bonds between nucleotide subunits of DNA, e.g. genomic DNA or mitochondrial DNA, to create a single or double strand break. Targeted integration can be promoted both by the presence of homology arms on the donor DNA flanking the integration site, and by contacting target cells with a donor DNA in the presence of—or after contacting the target DNA with—a targeted nuclease. By a “targeted nuclease” it is meant a nuclease that cleaves a specific DNA sequence to produce a double strand break at that sequence. In these aspects of the method, this cleavage site becomes the site of integration for the one or more genes of interest. As used herein, a nuclease includes naturally occurring nucleases as well as recombinant, i.e. engineered, nucleases.

One example of a targeted nuclease that may be used in the subject methods is a zinc finger nuclease or “ZFN”. ZFNs are targeted nucleases comprising a nuclease fused to a zinc finger DNA binding domain By a “zinc finger DNA binding domain” or “ZFBD” it is meant a polypeptide domain that binds DNA in a sequence-specific manner through one or more zinc fingers. A zinc finger is a domain of about 30 amino acids within the zinc finger binding domain whose structure is stabilized through coordination of a zinc ion. Examples of zinc fingers include C₂H₂ zinc fingers, C₃H zinc fingers, and C4 zinc fingers. A “designed” zinc finger domain is a domain not occurring in nature whose design/composition results principally from rational criteria, e.g. application of substitution rules and computerized algorithms for processing information in a database storing information of existing ZFP designs and binding data. See, for example, U.S. Pat. Nos. 6,140,081; 6,453,242; and 6,534,261; see also WO 98/53058; WO 98/53059; WO 98/53060; WO 02/016536 and WO 03/016496. A “selected” zinc finger domain is a domain not found in nature whose production results primarily from an empirical process such as phage display, interaction trap or hybrid selection. ZFNs are described in greater detail in U.S. Pat. Nos. 7,888,121 and 7,972,854, the complete disclosures of which are incorporated herein by reference. The most recognized example of a ZFN in the art is a fusion of the Fold nuclease with a zinc finger DNA binding domain.

Another example of a targeted nuclease that finds use in the subject methods is a TAL Nuclease (“TALN”, TAL effector nuclease, or “TALEN”). A TALN is a targeted nuclease comprising a nuclease fused to a TAL effector DNA binding domain By “transcription activator-like effector DNA binding domain”, “TAL effector DNA binding domain”, or “TALE DNA binding domain” it is meant the polypeptide domain of TAL effector proteins that is responsible for binding of the TAL effector protein to DNA. TAL effector proteins are secreted by plant pathogens of the genus Xanthomonas during infection. These proteins enter the nucleus of the plant cell, bind effector-specific DNA sequences via their DNA binding domain, and activate gene transcription at these sequences via their transactivation domains. TAL effector DNA binding domain specificity depends on an effector-variable number of imperfect 34 amino acid repeats, which comprise polymorphisms at select repeat positions called repeat variable-diresidues (RVD). TALENs are described in greater detail in US Patent Application No. 2011/0145940; in Christian, M et al. (2010) Targeting DNA Double-Strand Breaks with Tal Effector Nucleases. Genetics 186:757-761; and in Li, T. et al. (2010) TAL nucleases (TALNs): hybrid proteins composed of TAL effectors and FokI DNA-cleavage domain. Nucleic Acids Res. 39(1):359-372; the complete disclosures of which are incorporated herein by reference. The most recognized example of a TALEN in the art is a fusion polypeptide of the FokI nuclease to a TAL effector DNA binding domain.

Another example of a targeted nuclease that finds use in the subject methods is a targeted Spo11 nuclease, a polypeptide comprising a Spo11 polypeptide having nuclease activity fused to a DNA binding domain, e.g. a zinc finger DNA binding domain, a TAL effector DNA binding domain, etc. that has specificity for a DNA sequence of interest. See, for example, U.S. Application No. 61/555,857, the disclosure of which is incorporated herein by reference.

Other nonlimiting examples of targeted nucleases include naturally occurring and recombinant nucleases, e.g. restriction endonucleases, meganucleases homing endonucleases, CRISPR/Cas effector proteins (e.g., CRISPR/Cas endonucleases such as Cas9, Cas12, Cas13, and the like). Any targeted nuclease(s) that are specific for the integration site of interest and promote the cleavage of an integration site may be used. The targeted nuclease(s) may be stably expressed by the cells. Alternatively, the targeted nuclease(s) may be transiently expressed by the cells, e.g. it may be provided to the cells prior to, simultaneously with, or subsequent to contacting the cells with donor polynucleotide. If transiently expressed by the cells, the targeted nuclease(s) may be provided to cells as DNA. Alternatively, targeted nuclease(s) may be provided to cells as mRNA encoding the targeted nuclease(s), e.g. using well-developed transfection techniques; see, e.g. Angel and Yanik (2010) PLoS ONE 5(7): e11756; Beumer et al. (2008) PNAS 105(50):19821-19826, and the commercially available TransMessenger® reagents from Qiagen, Stemfect™ RNA Transfection Kit from Stemgent, and TranslT®-mRNA Transfection Kit from Minis Bio LLC. Alternatively, the targeted nuclease(s) may be provided to cells as a polypeptide. Such polypeptides may optionally be fused to a polypeptide domain that increases solubility of the product, and/or fused to a polypeptide permeant domain to promote uptake by the cell. The targeted nuclease(s) may be produced by eukaryotic cells or by prokaryotic cells, it may be further processed by unfolding, e.g. heat denaturation, DTT reduction, etc. and may be further refolded, using methods known in the art. It may be modified, e.g. by chemical derivatization or by molecular biology techniques and synthetic chemistry, e.g. to so as to improve resistance to proteolytic degradation or to optimize solubility properties or to render the polypeptide more suitable as a therapeutic agent.

Many examples of nucleases are known in the art, including Zinc finger nucleases (ZFNs), Transcription Activator-Like Effector Nucleases (TALENs), CRISPR/Cas effector proteins, meganucleases, homing endonucleases, restriction endonucleases, and the like (e.g., RecBCD endonuclease, T7 endonuclease, T4 endonuclease IV, Bal 31 endonuclease, Endonuclease I (endo I), Endonuclease II (endo VI, exo III), Micrococcal nuclease, Neurospora endonuclease, S1-nuclease, P1-nuclease, Mung bean nuclease I, Ustilago nuclease, Dnase I, AP endonuclease, EndoR, etc.). By an exogenous nuclease, it is meant a nuclease that comes from the outside of the cell, for example, a nuclease or a nucleic acid encoding a nuclease that is present and active in a living cell but that originated outside of that cell. As noted elsewhere herein, targeted genome editing in a cell can also be achieved without providing nucleases to the cell, i.e. without contacting the cell with nuclease or a nucleic acid encoding a nuclease.

### Kits/Systems

The present disclosure provides kits/systems for carrying out a subject method. In some embodiments a subject kit includes a ribonucleotide reductase inhibitor and a recombinant adeno-associated virus (rAAV) vector comprising a donor DNA for homologous recombination. In some cases the kit further includes a population of eukaryotic (e.g., mammalian, primate, non-human primate, human) target cells. A kit can further include one or more additional reagents, where such additional reagents can be any convenient reagent. Components of a subject kit can be in separate containers; or can be combined in a single container. In some cases one or more of a kit's components are pharmaceutically formulated for administration to a human.

In addition to above-mentioned components, a subject kit can further include instructions for using the components of the kit to practice the subject methods. The instructions for practicing the subject methods are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging) etc. In other embodiments, the instructions are present as an electronic storage data file present on a suitable computer readable storage medium, e.g. CD-ROM, diskette, flash drive, etc. In yet other embodiments, the actual instructions are not present in the kit, but means for obtaining the instructions from a remote source, e.g. via the internet, are provided. An example of this embodiment is a kit that includes a web address where the instructions can be viewed and/or from which the instructions can be downloaded. As with the instructions, this means for obtaining the instructions is recorded on a suitable substrate.

### Example Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of ordinary skill in the art upon reading this disclosure, each of the individually numbered aspects may be used or combined with any of the preceding or following individually numbered aspects. This is intended to provide support for all such combinations of aspects and is not limited to combinations of aspects explicitly provided below. It will be apparent to one of ordinary skill in the art that various changes and modifications can be made without departing from the spirit or scope of the invention.

1. A method of promoting homologous recombination for gene insertion, the method comprising: contacting a population of cells with:
(a) a ribonucleotide reductase inhibitor; and
(b) a recombinant adeno-associated virus (rAAV) comprising a donor DNA that comprises a sequence cassette flanked by homology arms, wherein the sequence cassette comprises a transgene sequence,
wherein the homology arms of the donor DNA facilitate integration of the sequence cassette into a genomic locus.
   2. The method of 1, wherein the transgene sequence is a protein-coding sequence.
   3. The method of 1, wherein the transgene sequence encodes a non-coding RNA.
   4. The method of any one of 1-3, wherein said sequence cassette further comprises a promoter that is operably linked to the transgene sequence.
   5. The method of any one of 1-3, wherein:
said sequence cassette further comprises a sequence, positioned 5′ or 3′ to the transgene sequence, that promotes production of two independent gene products upon integration of said sequence cassette into the genomic locus,
wherein the genomic locus comprises an endogenous gene and said sequence cassette integrates into the genomic locus such that after integration, the transgene sequence and the endogenous gene are both expressed under control of the endogenous gene's promoter without significantly disrupting expression of the endogenous gene.
   6. The method of 5, wherein the nucleotide sequence that promotes production of two independent gene products encodes a 2A peptide, an IRES, an intein, a recognition sequence for a site specific protease, a cleavable linker that is cleaved as part of the coagulation cascade, a factor XI cleavage site, or an intronic splice donor/splice acceptor sequence.
   7. The method of 6, wherein the nucleotide sequence that promotes production of two independent gene products encodes a 2A peptide.
   8. The method of any one of 1-7, wherein the method does not include delivering a nuclease or nucleic acid encoding a nuclease to the population of cells.
   9. The method of any one of 1-7, wherein the method includes delivering a site-specific nuclease or a nucleic acid encoding the site-specific nuclease to the population of cells.
   10. The method of 9, wherein the site-specific nuclease is a CRISPR/Cas effector protein, a Zinc Finger Nuclease (ZFN), a TALEN, or a meganuclease.
   11. The method of any one of 1-10, wherein the population of cells is in vitro or ex vivo.
   12. The method of 11, wherein the population of cells is contacted with the ribonucleotide reductase inhibitor for a period of time in a range of from 3-16 hours prior to contact with the rAAV.
   13. The method of any one of 1-10, wherein the population of cells is in vivo.
   14. The method of 13, wherein the ribonucleotide reductase inhibitor is administered to an individual at a dose in a range of from 0.5 to 100 milligrams per kilogram body weight (mpk).
   15. The method of 13, wherein the ribonucleotide reductase inhibitor is administered to an individual at least once a day for two or more consecutive days.
   16. The method of any one 1-15, wherein the ribonucleotide reductase inhibitor comprises an siRNA that targets ribonucleotide reductase.
   17. The method of any one 1-15, wherein the ribonucleotide reductase inhibitor comprises one or more compounds selected from the group consisting of: hydroxyurea (HU), motexafin gadolinium, fludarabine, cladribine, gemcitabine, tezacitabine, triapine, and gallium maltolate.
   18. The method of any one 1-15, wherein the ribonucleotide reductase inhibitor comprises fludarabine.
   19. The method of 18, wherein:
the population of cells is in vitro or ex vivo; and
the fludarabine is at a concentration in a range of from 20 μM to 500 μM.
   20. The method of 18. wherein:
the population of cells is in vitro or ex vivo; and
the fludarabine is at a concentration in a range of from 50 μM to 200 μM.
   21. The method of any one 1-15, wherein the ribonucleotide reductase inhibitor comprises hydroxyurea (HU).
   22. The method of 21, wherein:
the population of cells is in vitro or ex vivo; and
the HU is at a concentration in a range of from 0.5 mM to 30 mM.
   23. The method of 21, wherein:
the population of cells is in vitro or ex vivo; and
the HU is at a concentration in a range of from 4 mM to 10 mM.
   24. The method of any one 1-15, wherein the ribonucleotide reductase inhibitor comprises Gemcitabine.
   25. The method of 24, wherein:
the population of cells is in vitro or ex vivo; and
the Gemcitabine is at a concentration in a range of from 20 nM to 200 nM.
   26. The method of any one 1-25, wherein the sequence cassette integrates into two chromosomes such that the integration is homozygotic.
   27. A kit comprising:
(1) a ribonucleotide reductase inhibitor; and
(2) a recombinant adeno-associated virus (rAAV) comprising a donor DNA for homologous recombination.
   28. The kit of 27, further comprising (3) a population of eukaryotic cells.
   29. The kit of 28, wherein the population of eukaryotic cells is a population of mammalian cells.
   30. The kit of any one of 27-29, wherein the ribonucleotide reductase inhibitor comprises fludarabine, gemcitabine, hydroxyurea (HU), or any combination thereof.
   31. The kit of any one of 27-30, wherein the ribonucleotide reductase inhibitor and/or the rAAV is formulated for administration to an individual.
   32. The kit of any one of 27-30, wherein the ribonucleotide reductase inhibitor and/or the rAAV is pharmaceutically formulated for administration to a human.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., bp, base pair(s); kb, kilobase(s); pl, picoliter(s); s or sec, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Ribonucleotide Reductase Inhibition Enhanced Homologous Recombination Efficiency in Cells In Vitro

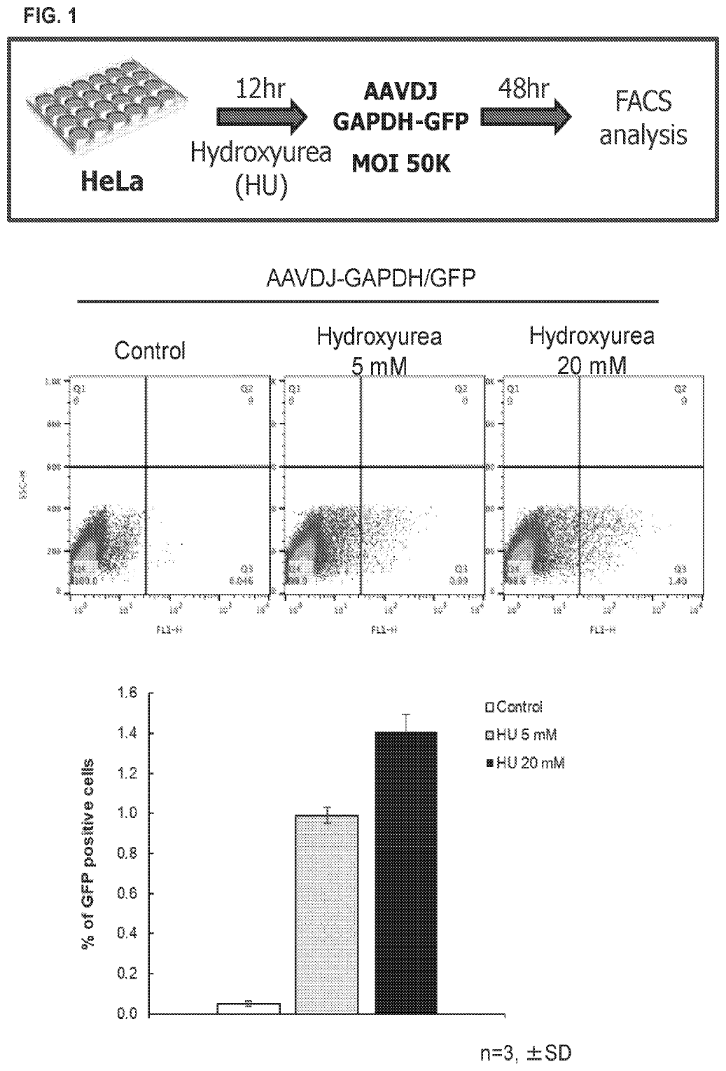

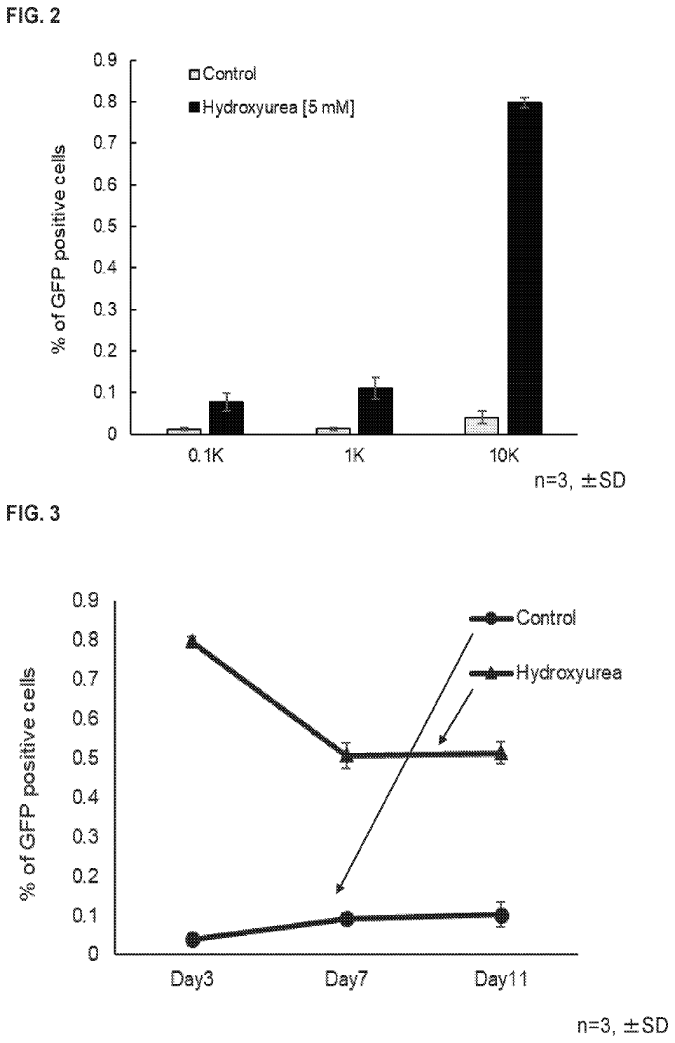

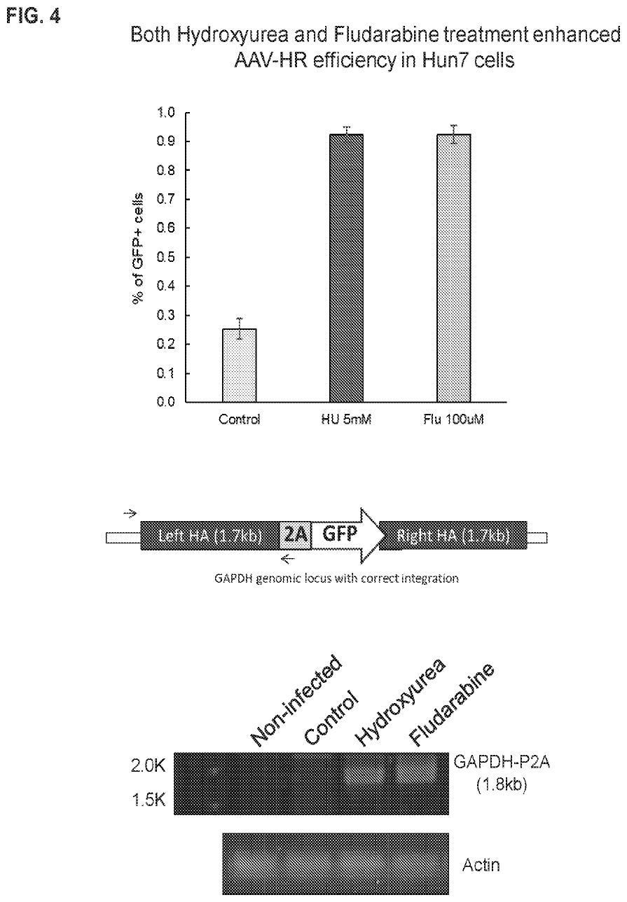

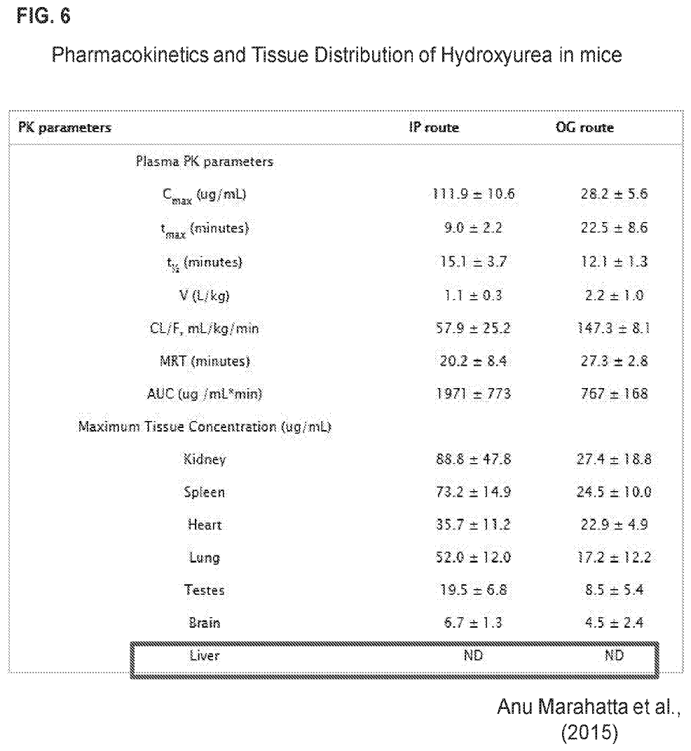

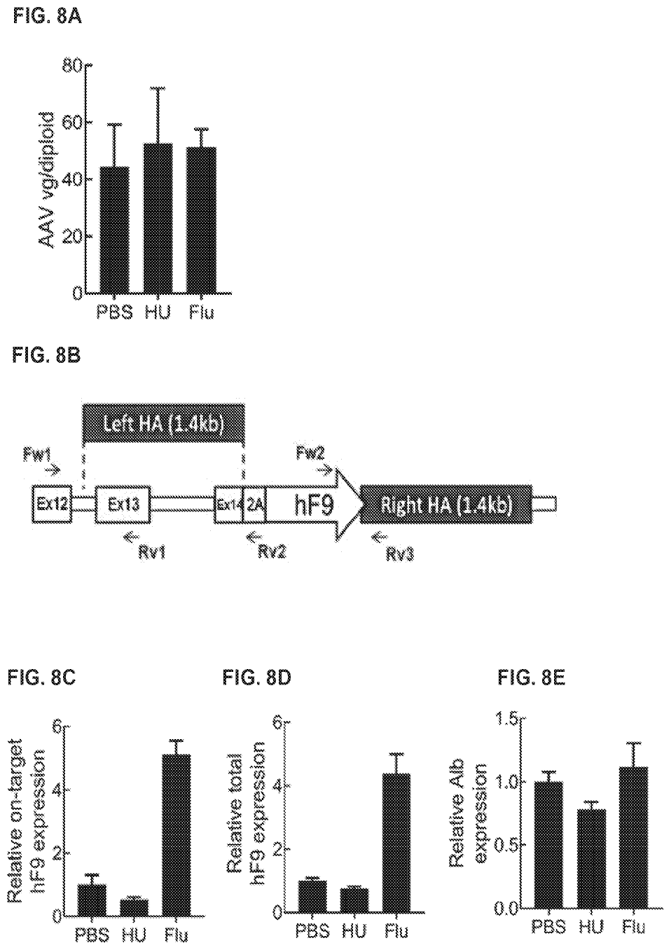

There are some small molecule compounds which have been reported to enhance or inhibit recombinant adeno-associated virus (rAAV) transduction. However, whether any of these compounds can affect the efficiency of AAV-mediated homologous recombination (HR) was unknown. To test this, several compounds (e.g., proteasome inhibitors, HDAC inhibitors, DNA methyltransferase inhibitors, DNA damage inducers, and so on) with different mechanisms of action were selected and tested for their effect on transduction and HR. As previously reported, all of the tested compounds reproducibly enhanced rAAV transduction in vitro using several different serotypes. The effect of the compounds in AAV-mediated HR was tested using an assay that employed an AAV-DJ vector that incorporates a GFP-coding sequence into the human GAPDH locus (a GAPDH targeting vector) of Huh7 cells. A 2A peptide sequence was present 5′ of the GFP coding sequence such that once integrated the 2A peptide sequence was present between the endogenous GAPDH coding sequence and the introduced GFP coding sequence. The only compounds that successfully and reproducibly increased HR efficiency (as indicated by a significantly increased GFP positive population by FACS analysis) were ribonucleotide reductase (RNR) inhibitors such as hydroxyurea, fludarabine and gemcitabine (FIG. 1, FIG. 2, FIG. 3, FIG. 4, FIG. 5, FIG. 7). In order to exclude the possibility that GFP expression came from episomal vectors, cells were cultured more than 10 days after AAV infection. Even after long term culture, the GFP positive population remained to be significantly higher in cells treated with RNR inhibitors than control cells (FIG. 3). Because RNR inhibitors are known to induce DNA damage, the effect of teniposide (another DNA damage agent) was also evaluated on AAV-HR efficiency. Teniposide showed no effect (FIG. 7). Given that hydroxyurea, fludarabine and gemcitabine have different mechanism of action for RNR inhibition, and all three compounds enhance AAV-HR efficiency, it was concluded that RNR inhibition enhances AAV-HR efficiency.

### Example 2: Ribonucleotide Reductase Inhibition Enhanced Homologous Recombination Efficiency in Cells In Vivo

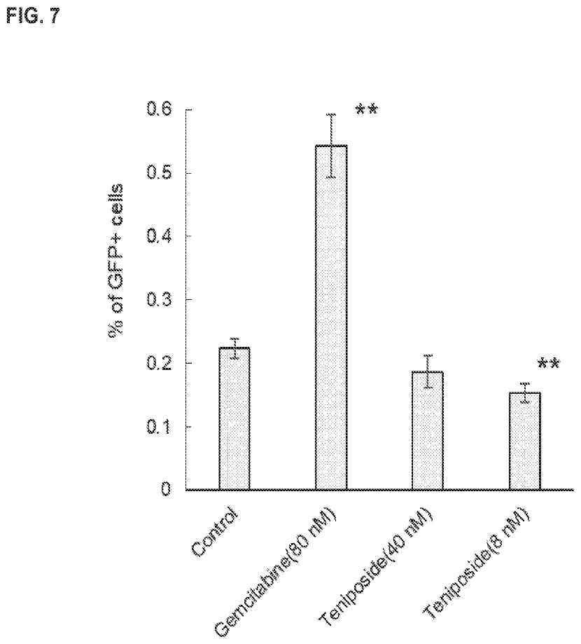

The efficacy of RNR inhibitors in vivo was tested using an AAV8 vector that incorporates a Alb/F9 targeting sequence. The “Alb/F9” targeting vector was an AAV that included a donor DNA having a nucleotide sequence of interest flanked by homology arms that facilitated integration of the sequence of interest into the genomic albumin (Alb) locus. The nucleotide sequence of interest in this case was a 2A peptide sequence 5′ of a sequence encoding human coagulation factor IX (hF9). Thus, once integrated into the genomic locus, the 2A peptide sequence was present between the endogenous albumin coding sequence and the introduced/integrated hF9 coding sequence such that expression of hF9 mRNA was under the control of the albumin promoter but translation of the transcript led to two separate proteins (albumin and hF9). Thus, integration/expression of hF9 into/from the albumin locus did not disrupt expression of albumin from the locus. Although hydroxyurea did not exhibit enhancement of hF9 expression, fludarabine treatment significantly and consistently increased serum hF9 levels without any body weight loss (FIG. 5). It is likely that hydroxyurea exhibited no effect because it is quickly metabolized in the liver and its liver distribution is quite low (FIG. 6). Thus, the lack of efficacy observed based on hF9 liver expression (see FIG. 5) is likely due to low/substantially no HU in the liver (FIG. 6). Given the in vitro HU results and the in vitro and in vivo fludarabine results—in addition to the gemcitabine results of FIG. 7, it is likely and expected that AAV-based HR is enhanced in vivo by HU in organs/tissues other than the liver (e.g., kidney, spleen, heart, lung, testes, brain, etc.).

### Example 3

### Results

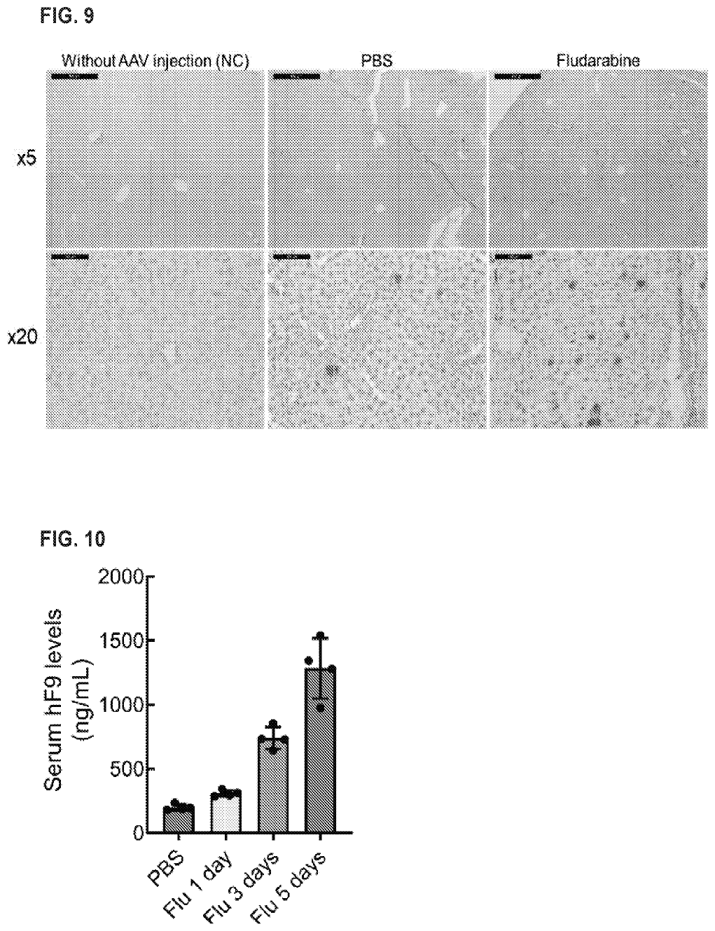

To determine if the increased hF9 expression shown in FIG. 5 was a result of higher transduction and vector copy number in liver, DNA extracted from the liver of treated animals was analyzed via qPCR. The copy number of rAAV vector genomes per diploid hepatocytes showed no significant change between the groups with or without drug administration (FIG. 8A). This finding suggested that increased vector transduction could not account for the significant differences in transgene expression and that fludarabine likely increased gene targeting positive cells in the mouse liver. To further investigate the basis of increased hF9 expression resulting from fludarabine administration, mRNA extracted from liver tissues of mice was analyzed using a primer set detecting Alb-P2A-hF9 fusion mRNA derived from on-target integration (FIG. 8B). The expression levels of this fusion mRNA were significantly higher in the fludarabine-treated group compared to the HU and PBS-treated groups (5.1-fold higher vs. PBS), consistent with the differences in hF9 protein levels and suggestive of increased gene targeting rate with fludarabine treatment (FIG. 8C). Examination of total hF9 mRNA, which includes fusion mRNA from targeted integration but also mRNA from any random integration- or episomal-derived expression, by RT-qPCR with a vector-internal primer set showed 4.4-fold increased levels in fludarabine treated mice compared to controls (FIG. 8D). Additionally, endogenous albumin expression was not changed by fludarabine treatment (FIG. 8E). These data suggested that fludarabine increases the efficiency of gene targeting in vivo.

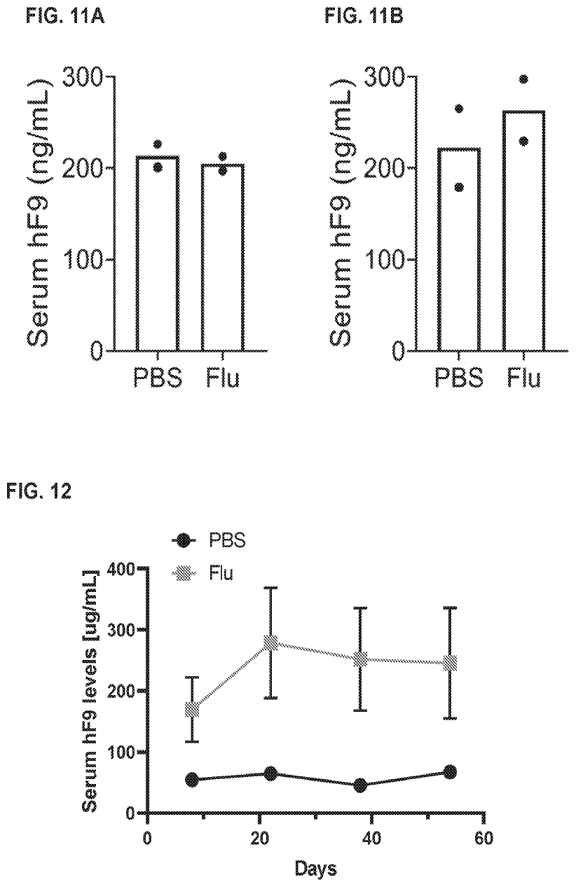

To ascertain mRNA transcription profiles on a single cell basis in mouse liver following rAAV and fludarabine treatment, RNAscope in situ hybridization was used to label hF9 mRNA. Liver tissue sections from Alb-P2A-hF9 injected mice showed hF9 mRNA was primarily expressed in very few hepatocytes (FIG. 9). The hF9-recognizing probe showed no staining in liver sections from a no rAAV injected mouse, ensuring specificity for hF9 mRNA. hF9 mRNA in liver of a fludarabine-treated mouse displayed strong detection in individual cells similar to the PBS treated mouse. Yet, the frequency of positive cells was clearly more frequent in the fludarabine-treated mouse. Collectively, these data demonstrated that fludarabine increased hF9 expression, through enhanced rAAV-mediated gene targeting efficiency, and did not increase vector-borne episomal transgene expression in mouse livers.

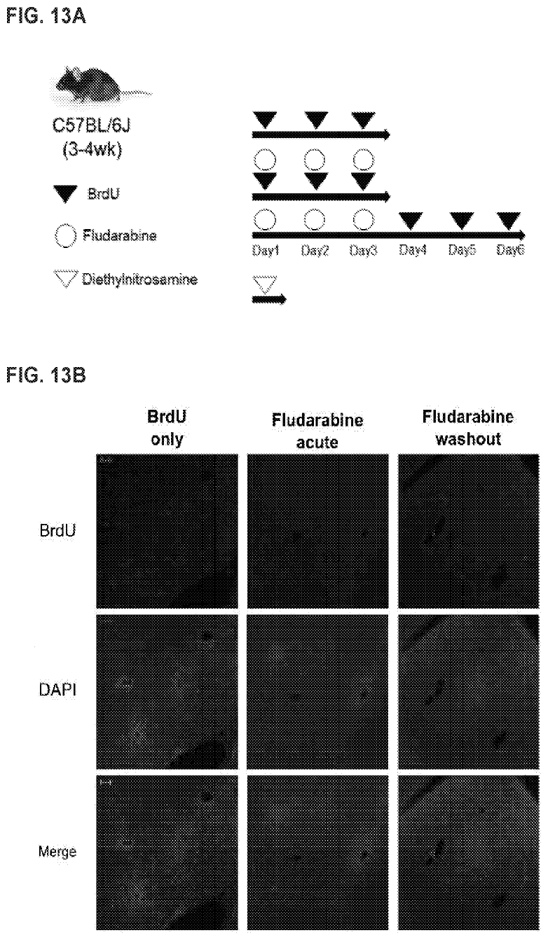

Next, whether the effect of fludarabine on gene targeting in mice can be modulated by dose was investigated by administrating fludarabine for 1, 3, or 5 sequential days. After injection of rAAV-Alb-P2A-hF9 on the first day of drug treatment, followed by sequential days of drug treatment, a clear increase in serum hF9 levels with increasing days of fludarabine injection was observed (FIG. 10). Five days of fludarabine treatment provided the highest hF9 levels (6.4-fold vs. PBS) significantly greater than three or one day of treatment. Interestingly, even a single administration of fludarabine showed about 2-fold greater levels of hF9 (FIG. 10).

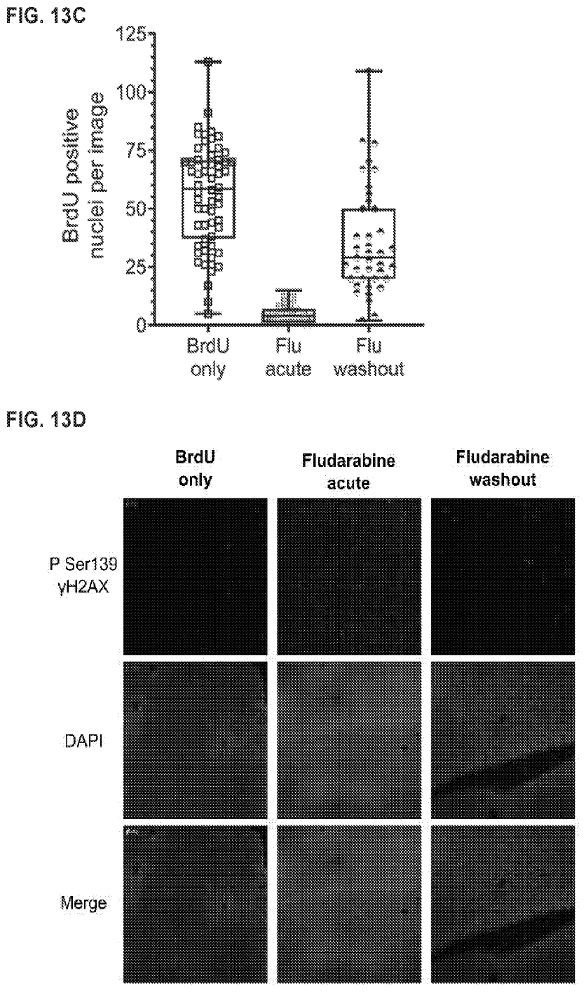

After determining that fludarabine's effect on gene targeting was affected by dose, whether the effect was also affected by time of dosing was addressed. As shown in this study, and from additional observations, stable and maximum hF9 levels are achieved around 2-3 weeks after injection of gene targeting rAAV vectors in mice (see, e.g., FIG. 5). This trend suggests that majority of AAV gene targeting likely occurs during the first two weeks post-injection. Therefore, the assumption was made that fludarabine should be administered during early time points in order to show efficacy on increasing gene targeting. To test this hypothesis, four mice were injected with rAAV8 packaged Alb-P2A-hF9 targeting vector and three weeks later divided into two different groups, based on the serum hF9 levels to create two groups of equal average hF9 levels (FIG. 11A). Then, PBS or fludarabine was administered for 3 days through days 28-30, followed by serum sample collection at day 54 (FIG. 11B). At this time point, fludarabine administration failed to alter hF9 expression as serum levels remained nearly identical between the PBS and fludarabine-treated mice. As such, fludarabine showed little to no effect on hF9 expression and presumably gene targeting efficiency, when given as a delayed dose. It is concluded that the enhancing effect or mechanisms of fludarabine on gene targeting efficiency is observed with temporal proximity to the early intracellular interactions of gene targeting AAV, occurring shortly after transduction, and not when transduction occurs weeks afterward.

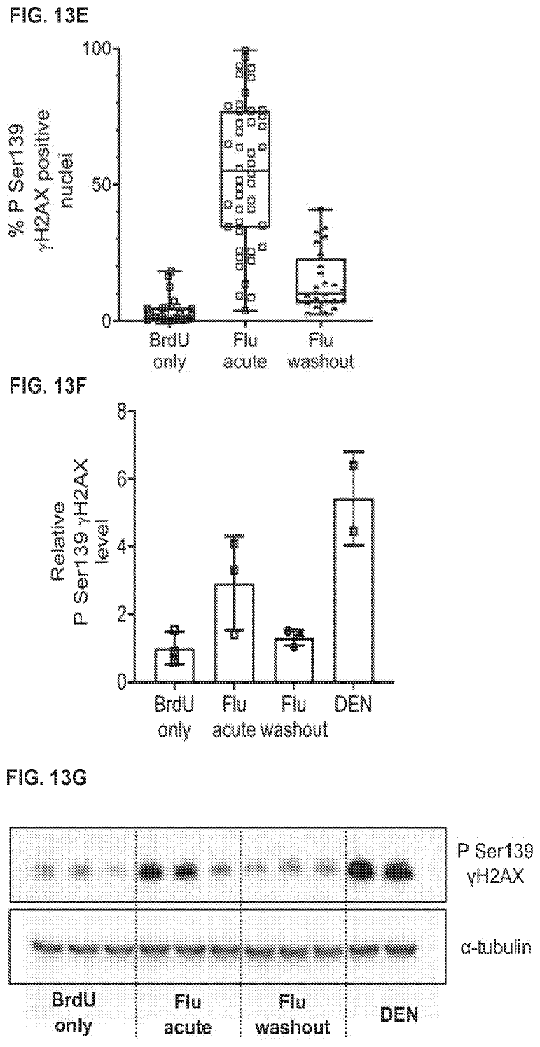

Also examined was whether fludarabine-mediated enhancement of gene targeting was Albumin specific—this was done by designing a vector targeting an alternative genomic locus, ApoE. ApoE is highly and specifically expressed in hepatocytes, similar to Albumin. When 1.0×10¹¹ vg/mouse of this vector (ApoE-P2A-hF9) was injected with fludarabine treatment, a significant increase in hF9 expression compared to PBS-injected mice was observed (FIG. 12). The fold-increase (3.6-fold at day 54) was similar to findings targeting the Albumin locus. This shows that fludarabine can strongly enhance AAV gene targeting in vitro and in vivo using different transgenes and differing genomic targets—and thus the methods and compositions described herein are useful for any desired target locus using any desired transgene.

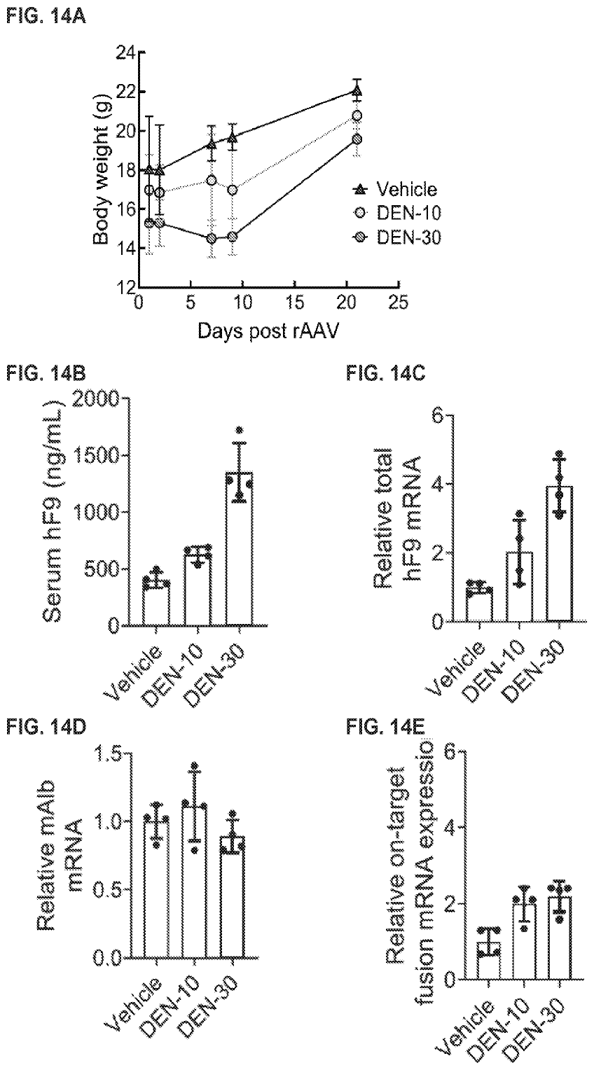

Fludarabine is known to inhibit the catalytic subunit of the RNR complex, RRM1 functioning throughout the cell cycle, which is mechanistically distinguished from another RNR inhibitor, hydroxyurea that specifically targets S phase by inhibiting RRM2, a S-phase-specific subunit of RNR complex. Since RNR catalyzes a critical step of dNTPs synthesis which is required for not only DNA replication but also to repair DNA lesions regardless of cell cycle, fludarabine can antagonize basal level DNA repair in non-dividing cells, such as in vivo hepatocytes, by decreasing dNTP pool. Moreover, fludarabine, as a purine analogue, is known to be incorporated into replicating DNA and also into nascent RNA strand, which leads to inhibit transcription. In order to determine the effect of fludarabine on in vivo hepatocytes, proliferation and DNA damage signaling was investigated by analyzing BrdU incorporation and γH2AX expression levels. First, BrdU was injected either during the time of fludarabine treatment or in a washout phase (i.e. post-drug administration), to determine the acute and long-term effect of the drug on proliferation, respectively (FIG. 13A). In addition to the BrdU injections, the first group received mock treatment (PBS) and the second group received 125 mg/kg fludarabine, each given three times per day for three days Similarly, the last group received the three doses for three days of fludarabine (without BrdU) but was followed by three days of BrdU labeling Animals were sacrificed shortly after the last BrdU injection and liver tissues were harvested for immunohistochemistry (IHC) staining of BrdU incorporated DNA using an antibody against BrdU. BrdU labeled nuclei were nearly absent in liver sections taken immediately after fludarabine administration, demonstrating a strong inhibitory effect of fludarabine on hepatocyte proliferation in vivo similar to in vitro reports (FIGS. 13B and C). BrdU incorporation during the washout phase of fludarabine treated mice was significantly greater than BrdU incorporation in the acute drug administration phase, suggesting these BrdU positive hepatocytes progressed through S phase after the drug washout (FIGS. 13B and C). IHC staining of phosphorylated Ser139 γH2AX (P Ser139 γH2AX) was then performed since phosphorylation of the Serine139 residue on the histone γH2AX occurs in response to a variety of DNA damage and is well accepted biomarker of damage-dependent kinase activity. Mice were also treated with diethylnitrosamine (DEN), as a positive control to induce DNA damage signaling in liver since DEN is known to be genotoxic in the liver of various animals. DEN is an alkylating agent that has considerable genotoxicity in the liver and has been used to generate de novo animal models of hepatocellular carcinomas. γH2AX was rarely detected in BrdU/PBS treated mouse liver tissues, with the few positive nuclear localized foci likely representing background or basal levels of DNA damage in the homeostasis condition of liver (FIGS. 13D and E). Liver sections from DEN-treated mice showed intense nuclear staining of γH2AX consistent with previous reports, confirming that immunohistochemistry staining could detect DNA damage response in mouse liver. γH2AX was nearly absent in control animal liver. In clear contrast, during the time proximate to fludarabine treatment mouse livers possessed widespread nuclear localized γH2AX foci (FIGS. 13D and E). In the three-day washout time period after drug administration had stopped, γH2AX foci became significantly reduced compared to immediately proximal to fludarabine treatment. These findings were consistent with Western blotting for γH2AX in liver tissue lysates, from the same animals, whereby strong detection of γH2AX was seen in the acute phase of fludarabine treated sample (FIGS. 13F and G). Importantly, the number of γH2AX foci positive hepatocytes were much greater than BrdU positive ones (FIGS. 13B and D), implicating that fludarabine treatment likely induced DNA damage signaling in both proliferating and non-proliferating hepatocytes.

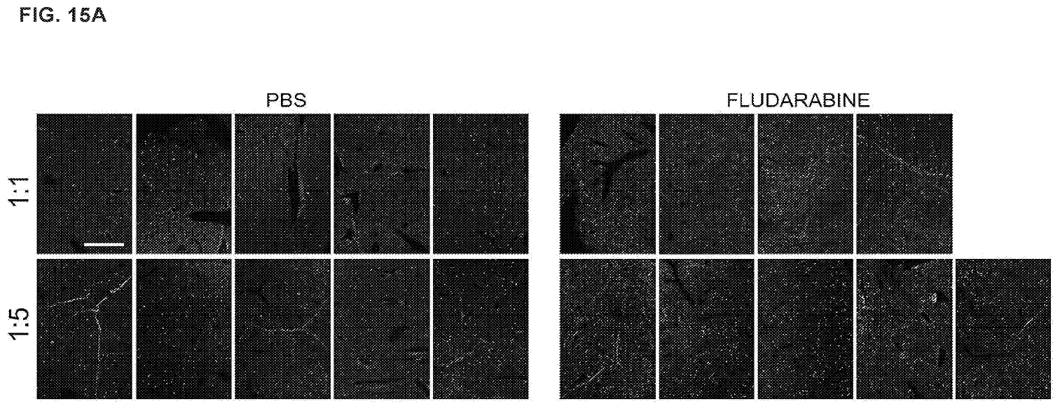

In order to examine the importance of DNA damage response and also the potential for genotoxic treatments to enhance AAV gene targeting in vivo, DEN injections were utilized, whose strong genotoxic potential and high liver bioavailability made it suitable for testing genotoxicity's impact on AAV gene targeting. DEN administration decreased body weight gains compared to vehicle-injected mice in a dose-dependent manner (FIG. 14A). The highest dose of DEN (30 mg/kg) significantly increased serum hF9 protein levels (about 3.6-fold) compared to vehicle group three weeks post rAAV injection (FIG. 14B). Low dose administration (10 mg/kg) showed a trend of increased hF9 levels (˜1.5-fold), although it was not statistically significant, qPCR analysis using mRNA extracted from the liver tissue of these mice showed that the expression of total hF9 mRNA was increased by DEN administration (2- and 3.9-fold at low and high dose, respectively), consistent with ELISA data of serum hF9 (FIG. 14C). Endogenous albumin mRNA expression was nearly unchanged among all experimental groups (FIG. 14D). qPCR analysis to specifically detect on-target fusion Alb-P2A-hF9 mRNA, performed as described in prior experiments, showed only a modest increase of on-target HR derived fusion mRNA in DEN treated groups, of about 2-fold in both dose groups (FIG. 14E). These data did not correlate with total hF9 mRNA levels and implied that increased total hF9 mRNA and hF9 protein by DEN administration resulted from enhanced random integration, with minor increases in on-target HR. Thus, DEN treatment failed to enhance gene targeting to the extent as did fludarabine, suggesting the type of DNA damage or which repair pathway is activated might be important for optimal gene targeting enhancement. Taken all together, the data suggest that the class of RNR inhibitors, such as fludarabine can safely increase rAAV-mediated gene targeting efficiency in hepatocytes through induction of DNA repair signaling. Furthermore, fludarabine and similar drugs can expand the clinical applications of rAAV-mediated gene editing therapy in the absence of exogenous nucleases.

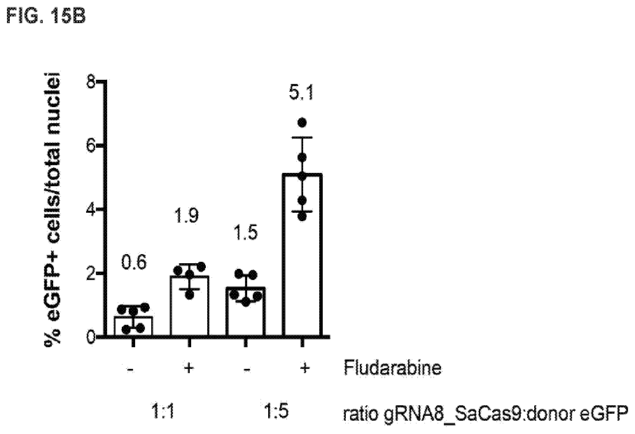

Lastly, it was investigated whether fludarabine administration can also increase the efficiency of rAAV-mediated gene targeting in coupled with programmable CRISPR/Cas9 which induces DNA double strand break at the locus of gene targeting. In this experiment, 2 different rAAV8 vectors were simultaneously injected: one contained Alb-P2A-GFP targeting vector whose sequence was same as promoterless hF9 vector except for GFP sequence instead of hF9 at 6.0×10¹² vg/kg or 3.0×10¹³ vg/kg, whereas the other one expressed both the SaCas9 and the sgRNA under the transcriptional control of liver-specific and U6 promoters at 6.0×10¹² vg/kg, respectively. The PAM sequence was located close to the stop codon of endogenous Albumin gene locus. Fluorescence GFP imaging analysis performed at 2 weeks after AAV injection clearly showed the increase of the number of GFP positive hepatocytes in fludarabine-treated mice compared with PBS-treated mice in both dose conditions (FIG. 15A). Quantification of the number of GFP positive hepatocytes further confirmed the results (FIG. 15B), suggesting that fludarabine treatment can enhance the gene targeting efficiency even in the presence of programmable DNA nuclease, such as CRISPR/Cas9.

### Material and Methods

### Vectors

The AAV vectors containing ITR sequences used in this study are based on AAV type 2 backbone. CAG-Fluc, Albumin-P2A-hF9, HLP-hAAT vectors were prepared as described previously (ref). Albumin-P2A-GFP vector was generated by replacing hF9 coding sequence of the Albumin-P2A-hF9 vector with GFP coding sequence using In-Fusion® HD Cloning Kit (TAKARA). SaCas9-sgRNA8 vector and Albumin-P2A-GFP vectors used were prepared as described previously (Caneva et al. 2019). For GAPDH-P2A-GFP vector construction, human genomic GAPDH segments were PCR-amplified using Fw: 5′-GACTGTACAGGGCTGCTCACATATTCTGG-3′ (SEQ ID NO: 1) and Rv: 5′-CTGTGTACAGAGTGTATGTGGCTGTGGCCC-3′ (SEQ ID NO: 2) (both containing BsRG1 sites for cloning) and inserted between AAV2 ITRs into BsrGI restriction sites in a modified pTRUF backbone (Grimm et al. 2006). The genomic segment spans approximately 1.7 Kb upstream and 1.7 kb downstream to the GAPDH stop codon. A 1,359 bp fragment was then synthesized spanning the region at the end of the GAPDH locus between the two SexA1 sites to be cloned in the vector. In this fragment, the GAPDH stop codon was removed and it was inserted an optimized P2A coding sequence preceded by a linker coding sequence (glycine-serine-glycine) and followed by the GFP sequence (without the start codon). For Apoe-P2A-hF9 vector construction, a genomic fragment containing sequences used for both homology arms was amplified from Blk6 mouse genomic DNA. Primers mApoE_10 F (5′-TCC ACA CCT GCC TAG TCT CG-3′) (SEQ ID NO: 3) and mApoE_10R (5′-GTG CCA GAG GCA GTT GAG TT-3′) (SEQ ID NO: 4) were used to amplify a 2.9 kb fragment. The PCR product was directly cloned into the pCR Blunt II TOPO vector using the Zero Blunt TOPO PCR cloning kit (Invitrogen), sequence verified, and used to generate the homology arms of the GeneRide vector. The left part was amplified from the cloned ApoE genomic fragment using primers ApoE_left_F (5′-ata tca tcg atc gcg atg cat taa tta agc ggc cgA AGA CTG TAG GTC CTG ACC C—3′) (SEQ ID NO: 5) and ApoE_left_R (5′-ggt ggc gcc get tcc TTG ATT CTC CTG GGC CAC-3′) (SEQ ID NO: 6), the middle part was amplified from a previously described Alb-FIX GeneRide vector (Barzel et al., 2015) using primers ApoE_F9_F (5′-gcc cag gag aat caa GGA AGC GGC GCC ACC AAT-3′) (SEQ ID NO: 7) and ApoE_F9_R (5′-gga gaa gga tac tca TGT CAG CTT GGT CTT TTC TTT GAT CC-3′) (SEQ ID NO: 8), the right part was amplified from the cloned ApoE genomic fragment using primers ApoE_right_F (5′-aag acc aag ctg aca TGA GTA TCC TTC TCC TGT CCT GC-3′) (SEQ ID NO: 9) and ApoE_right_R (5′-acg taa cag atc tga tat cac gcg tgt aca cta gtG CCC TGC TGA GTC CCT GAG-3′) (SEQ ID NO: 10). Phusion Hot Start Flex (NEB) was used for all amplifications. Amplicons were purified using the Qiaquick PCR purification kit (Qiagen) and assembled into an Eag I and Spe I pre-digested AAV2 ITR containing vector using the NEBuilder HiFi DNA Assembly Master Mix (NEB) according to instructions

### Mice AAV Injection, Drug Treatment, Bleeding and Tissue Sampling

B6 mice were received tail vain injections of rAAV8 packaging each vector at the designated dose and were bled at indicated time points. Body weight of mice were measured using Scout pro portable scale (Ohaus) at indicated time points. Serum samples were obtained by centrifuge at 10,000 rpm for 10 minutes and used for ELISA assay of hF9. Hydroxyurea or fludarabine was resuspended in PBS and were injected intraperitoneally with indicated dose/regimen. For BrdU labeling of proliferating mice hepatocytes, BrdU was resuspended in PBS and were intraperitoneally injected for indicated time periods at 200 mg/kg per day for 3 days or 7 days. Diethylnitrosamine solution was prepared using saline and was also intraperitoneally injected for 3 days at 10 or 30 mg/kg. At the end of experiments, mice were anesthetized with isoflurane and perfused transcardially with PBS and then liver tissues were quickly harvested and cut into several pieces. The tissues for mRNA extractions were immediately submerged in RNAlater solution (Sigma) and stored at 4° C. until use. For gDNA or protein extraction, tissues were snap-frozen in liquid nitrogen and stored at −80° C. until use.

### AAV Production

rAAV vectors were produced as previously described using a Ca3(PO4)2 transfection protocol followed by CsCl gradient purification (Grimm et al. 2006) or using AAVpro® Purification Kit (All Serotypes) purchased from Takara Bio. Purified rAAVs were stored at −80° C. until used. rAAV genomes were extracted and purified using QIAamp MinElute Virus Spin Kit (QIAGEN) and were titered by qPCR.

### Flow Cytometry

Huh7 cells were harvested and washed with cold PBS and resuspended in cold PBS containing 3% FBS. Cells were kept on ice and protected from light until analyzed. The number of GFP expressing cells was evaluated using the BD FACSCalibur™ instrument and data were analyzed using the FlowJo software package.

RNA Extraction and cDNA Preparation

Cultured cells were washed with PBS once and total RNA was extracted using RNeasy micro plus kit (QIAGEN) according to the manufacture's protocol with DNase treatment. Liver tissue samples stabilized in RNA later solution (−100 mg) were homogenized in RINO 1.5 mL Screw-Cap Tube filled with stainless steel beads and 600 μL of RLT buffer (including □-mercaptoethanol) using Bullet Blender. Total RNA was extracted from the tissue lysates using RNeasy plus mini kit (QIAGEN) with additional on-column DNase treatment. cDNA was synthesized from 200-500 ng of total RNA using High-Capacity RNA-to-cDNA™ Kit (Life Technologies) according to the manufacturer's instructions.

gDNA Extraction

Cultured cells were collected by trypsinization and washed with PBS. Then total genomic DNA was extracted using QIAamp DNA Mini Kit (QIAGEN) according to the manufacture's protocol with RNase A treatment. Snap-frozen liver tissue (−100 mg) were homogenized in RINO 1.5 mL Screw-Cap Tube filled with stainless steel beads and 600 μL of AL buffer using Bullet Blender. Total RNA was extracted from the tissue lysates using DNeasy Blood & Tissue Kit (QIAGEN).

PCR and qRT-PCR

The polymerase chain reactions (PCRs) to amplify genomic regions where homologous integrations occurred (junction PCR) were performed using Q5® Hot Start High-Fidelity 2X Master Mix (New England Biolabs). The following cycling conditions were used: Human □-Actin (one cycle of 98° C. for 30 sec, 28 cycles of 98° C. for 10 sec, 60° C. for 15 sec, and 72° C. for 10 sec, and one cycle of 72° C. for 2 min), GAPDH-P2A junction (one cycle of 98° C. for 30 sec, 35 cycles of 98° C. for 10 sec, 62° C. for 15 sec, and 72° C. for 1 min, and one cycle of 72° C. for 2 min), Mouse albumin (one cycle of 98° C. for 30 sec, 32 cycles of 98° C. for 10 sec, 60° C. for 10 sec, and 72° C. for 2 min, and one cycle of 72° C. for 2 min), hF9-Albumin junction nested PCR (one cycle of 98° C. for 30 sec, 20 cycles (1″ PCR) and 25 cycles (2^{nd} PCR) of 98° C. for 10 sec, 62° C. for 15 sec, and 72° C. for 1 min, and one cycle of 72° C. for 2 min). PCR products were analyzed in agarose gels containing Ethidium bromide and visualized using ChemiDoc Imaging Systems (Bio-Dad). All sequence information is listed in Table1.

QPCR was performed in duplicate using Apex qPCR GREEN Master Mix (Genesee Scientific) and CFX384 Touch Real-Time PCR Detection System (Bio-Rad) using the following cycling conditions: 95° C. for 15 min, 45 cycles of 95° C. for 10 sec, 60° C. for 10 sec and 72° C. for 10 sec, and one cycle of 95° C. for 10 sec and 65° C. for 1 min and 65-97° C. (05° C./sec). Standard curves for each primer set were made and used for quantification. CFX Maestro Software was used for data analysis and relative mRNA expression levels were calculated by normalized against β-actin.

### Protein Extraction and Western Blotting

Total cell lysates from mice liver tissues were prepared using RIPA buffer containing Halt™ Protease and Phosphatase Inhibitor Cocktail (both from Thermo Fisher). Liver tissues were homogenized in RINO 1.5 mL Screw-Cap Tube filled with stainless steel beads and 600 μL of RIPA buffer using Bullet Blender. Protein concentration were measured using Pierce™ BCA Protein Assay Kit (Thermo Fisher) and the same amount of proteins for each sample were loaded into NuPAGE™ 4-12% Bis-Tris Protein Gels (Thermo Fisher). iBlot2 transfer system (Thermo Fisher) was used for western blotting. PVDF membranes were blocked with 5% BSA containing TBS-T buffer and the following 1^{st} antibodies were used. HRP-conjugated anti-α-tubulin (CST, 1:2000) and anti-γH2AX (CST, 1:2000) antibodies. HRP-conjugated secondary antibodies were used, and signals were detected using Pierce™ ECL Plus Western Blotting Substrate (Thermo Fisher) and ChemiDoc Imaging Systems (Bio-Dad)

### Immunohistochemistry Staining of Liver Sections

For all in situ hybridization and immunostaining experiments liver tissue was dissected into 2-3 mm pieces and fixed for 24 hours in 10% neutral buffered formalin (Sigma Aldrich, St. Louis, Mo.) at 4° C. Tissue was subsequently processed through 10%, 20%, and 30% sucrose solutions for 24 hours each, then frozen embedded into OCT media (Sakura Finetek USA, Torrance, Calif.) with liquid nitrogen and 2-Methylbutane (Sigma Aldrich). Frozen tissue was sectioned into 16 μm thick sections using a Microm HM550 Microtome (Thermo Scientific, Waltham Mass.). Tissue sections were blocked with antibody diluent comprised of 5.0% normal donkey serum (Jackson Immuno Research, West Grove, Pa.) and 0.1% Triton-X 100 (Sigma Aldrich). GFP was stained with an anti-GFP chicken IgY primary antibody (Invitrogen, Carlsbad, Calif.) and phosphorylated Ser139 γH2AX was stained with a rabbit monocolonal (20E3) (Cell Signaling Technologies, Danvers, Mass.). Polyclonal secondary detection antibodies consisted of anti-chicken IgY antibody conjugated to Alexa Fluor 488 (Jackson Immuno Research) and polyclonal anti-rabbit IgG antibody conjugated to Alexa Fluor 594 (Thermo Scientific).

Detection of BrdU incorporated DNA was accomplished with heat denaturing in an antigen retrieval buffer (Advanced Cell Diagnostics, Newark, CA), followed by staining with a rat monoclonal anti-BrdU antibody (BU1/75 (ICR1)) (Abcam, Cambridge, UK) and secondary Alexa Fluor 594 antibody (Thermo Scientific). All IHC slides were mounted with Prolong Diamond Antifade with Dapi (Thermo Scientific) and imaged on a Zeiss LSM 880 confocal microscope. Specificity of all staining procedures was ensured with appropriate biological controls and control slides stained with secondary antibody only.

RNAscope In Situ Hybridization of hFIX

Liver tissue was processed for RNA in situ hybridization as described above. Fixed frozen tissue was sectioned into 9 μm thick sections and RNAScope hybridization was performed according to the manufacturer's protocol (Advanced Cell Diagnostics). A custom probe was designed to detect codon-optimized human factor IX mRNA. Slides were counterstained with 50% hematoxylin (Thermo Scientific). Imaging was performed using a Leica DM2000 brightfield microscope.

### Image Analysis

All image analysis was performed in a blinded manner Analysis of BrdU incorporation was performed manually requiring nuclear colocalization of BrdU signal and greater signal intensity over background to be recorded as a positive nucleus. Signal from overtly non-hepatocyte nuclei directly associated with larger liver structures such as central veins or bile ducts were not included in tally. Scoring of phosphorylated Ser139 γH2AX was performed using ImageJ software.

### Enzyme-Linked Immunosorbent Assay (ELISA)

Mice serum samples were used to quantify hF9 protein expression levels. ELISA for hF9 was performed as previously described with the following antibodies: mouse anti-hF9 IgG primary antibody at 1:1,000 (Sigma Cat#F2645), and polyclonal goat anti-hF9 peroxidase-conjugated IgG secondary antibody at 1:4,000 (Enzyme Research Cat#GAFIX-APHRP).

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

What is claimed is:

1. A method of promoting homologous recombination for gene insertion, the method comprising:
contacting a population of cells with:
(a) a ribonucleotide reductase inhibitor; and
(b) a recombinant adeno-associated virus (rAAV) comprising a donor DNA that comprises a sequence cassette flanked by homology arms, wherein the sequence cassette comprises a transgene sequence,
wherein the homology arms of the donor DNA facilitate integration of the sequence cassette into a genomic locus.

2. The method of claim 1, wherein the transgene sequence is a protein-coding sequence.

3. The method of claim 1, wherein the transgene sequence encodes a non-coding RNA.

4. The method of any one of claims 1-3, wherein said sequence cassette further comprises a promoter that is operably linked to the transgene sequence.

5. The method of any one of claims 1-3, wherein:
said sequence cassette further comprises a sequence, positioned 5′ or 3′ to the transgene sequence, that promotes production of two independent gene products upon integration of said sequence cassette into the genomic locus,
wherein the genomic locus comprises an endogenous gene and said sequence cassette integrates into the genomic locus such that after integration, the transgene sequence and the endogenous gene are both expressed under control of the endogenous gene's promoter without significantly disrupting expression of the endogenous gene.

6. The method of claim 5, wherein the nucleotide sequence that promotes production of two independent gene products encodes a 2A peptide, an IRES, an intein, a recognition sequence for a site specific protease, a cleavable linker that is cleaved as part of the coagulation cascade, a factor XI cleavage site, or an intronic splice donor/splice acceptor sequence.

7. The method of claim 6, wherein the nucleotide sequence that promotes production of two independent gene products encodes a 2A peptide.

8. The method of any one of claims 1-7, wherein the method does not include delivering a nuclease or nucleic acid encoding a nuclease to the population of cells.

9. The method of any one of claims 1-7, wherein the method includes delivering a site-specific nuclease or a nucleic acid encoding the site-specific nuclease to the population of cells.

10. The method of claim 9, wherein the site-specific nuclease is a CRISPR/Cas effector protein, a Zinc Finger Nuclease (ZFN), a TALEN, or a meganuclease.

11. The method of any one of claims 1-10, wherein the population of cells is in vitro or ex vivo.

12. The method of claim 11, wherein the population of cells is contacted with the ribonucleotide reductase inhibitor for a period of time in a range of from 3-16 hours prior to contact with the rAAV.

13. The method of any one of claims 1-10, wherein the population of cells is in vivo.

14. The method of claim 13, wherein the ribonucleotide reductase inhibitor is administered to an individual at a dose in a range of from 0.5 to 100 milligrams per kilogram body weight (mpk).

15. The method of claim 13, wherein the ribonucleotide reductase inhibitor is administered to an individual at least once a day for two or more consecutive days.

16. The method of claim any one claims 1-15, wherein the ribonucleotide reductase inhibitor comprises an siRNA that targets ribonucleotide reductase.

17. The method of claim any one claims 1-15, wherein the ribonucleotide reductase inhibitor comprises one or more compounds selected from the group consisting of: hydroxyurea (HU), motexafin gadolinium, fludarabine, cladribine, gemcitabine, tezacitabine, triapine, and gallium maltolate.

18. The method of claim any one claims 1-15, wherein the ribonucleotide reductase inhibitor comprises fludarabine.

19. The method of claim 18, wherein:
the population of cells is in vitro or ex vivo; and
the fludarabine is at a concentration in a range of from 20 μM to 500 μM.

20. The method of claim 18, wherein:
the population of cells is in vitro or ex vivo; and
the fludarabine is at a concentration in a range of from 50 μM to 200 μM.

21. The method of claim any one claims 1-15, wherein the ribonucleotide reductase inhibitor comprises hydroxyurea (HU).

22. The method of claim 21, wherein:
the population of cells is in vitro or ex vivo; and
the HU is at a concentration in a range of from 0.5 mM to 30 mM.

23. The method of claim 21, wherein:
the population of cells is in vitro or ex vivo; and
the HU is at a concentration in a range of from 4 mM to 10 mM.

24. The method of claim any one claims 1-15, wherein the ribonucleotide reductase inhibitor comprises Gemcitabine.

25. The method of claim 24, wherein:
the population of cells is in vitro or ex vivo; and
the Gemcitabine is at a concentration in a range of from 20 nM to 200 nM.

26. The method of any one claims 1-25, wherein the sequence cassette integrates into two chromosomes such that the integration is homozygotic.

27. A kit comprising:
(1) a ribonucleotide reductase inhibitor; and
(2) a recombinant adeno-associated virus (rAAV) comprising a donor DNA for homologous recombination.

28. The kit of claim 27, further comprising (3) a population of eukaryotic cells.

29. The kit of claim 28, wherein the population of eukaryotic cells is a population of mammalian cells.

30. The kit of any one of claims 27-29, wherein the ribonucleotide reductase inhibitor comprises fludarabine, gemcitabine, hydroxyurea (HU), or any combination thereof.

31. The kit of any one of claims 27-30, wherein the ribonucleotide reductase inhibitor and/or the rAAV is formulated for administration to an individual.

32. The kit of any one of claims 27-30, wherein the ribonucleotide reductase inhibitor and/or the rAAV is pharmaceutically formulated for administration to a human.
